(19) United States
(12) Patent Application Publication
(10) Pub. No.: US 2015/0376272 A1
(43) Pub. Date: Dec. 31, 2015

(54) INHIBITION OF ANGIOGENESIS IN REFRACTORY TUMORS

(71) Applicant: Genentech, Inc., South San Francisco, CA (US)

(72) Inventors: Alicia Chung, Berkeley, CA (US); Napoleone Ferrara, La Jolla, CA (US)

(73) Assignee: GENENTECH, INC., South San Francisco, CA (US)

(21) Appl. No.: 14/599,132

(22) Filed: Jan. 16, 2015

Related U.S. Application Data
(63) Continuation of application No. 14/179,882, filed on Feb. 13, 2014, now abandoned.
Application No. 14/179,882 is a continuation of application No. PCT/US2012/051220, filed on Aug. 16, 2012.
(60) Provisional application No. 61/524,670, filed on Aug. 17, 2011.

(51) Int. Cl. C07K 16/24; A61N 5/10; A61K 39/395; C07K 16/22; A61K 45/06

(52) U.S. Cl. CPC C07K 16/244; C07K 16/22; A61K 45/06; A61K 39/3955; C07K 16/243; A61N 5/10; C07K 2317/76; C07K 2317/24

(57) ABSTRACT

The present invention relates generally to the inhibition of tumor angiogenesis. In particular, the invention concerns the prevention or treatment of tumor angiogenesis and the suppression of tumor growth in tumors refractory to an anti-vascular endothelial growth factor (VEGF) treatment, using IL-17 antagonists, such as anti-IL-17 antibodies and other antagonists.

### RELATED APPLICATIONS

This application is a continuation of U.S. Ser. No. 14/179,882 filed Feb. 13, 2014, which is a continuation of International Patent Application No. PCT/US2012/051220, filed 16 Aug. 2012, which claims the benefit under 35 USC 119(e) of U.S. Provisional Application No. 61/524,670 filed 17 Aug. 2011, the contents of all of which are incorporated herein by reference.

### SEQUENCE LISTING

The instant application contains a Sequence Listing submitted via EFS-Web and hereby incorporated by reference in its entirely. Said ASCII copy is named P4729R1C2_US_Sequence_Listing.txt, was created on Jan. 15, 2015, and is 3,428 bytes in size.

### FIELD OF THE INVENTION

The present invention relates generally to the inhibition of tumor angiogenesis. In particular, the invention concerns the prevention or treatment of tumor angiogenesis and the suppression of tumor growth in tumors refractory to an anti-vascular endothelial growth factor (VEGF) treatment, using IL-17 antagonists, such as anti-IL-17 antibodies and other antagonists.

### BACKGROUND OF THE INVENTION

It is now well established that angiogenesis, which involves the formation of new blood vessels from preexisting endothelium, is implicated in the pathogenesis of a variety of disorders. These include solid tumors and metastasis, atherosclerosis, retrolental fibroplasia, hemangiomas, chronic inflammation, intraocular neovascular syndromes such as proliferative retinopathies, e.g., diabetic retinopathy, age-related macular degeneration (AMD), neovascular glaucoma, immune rejection of transplanted corneal tissue and other tissues, rheumatoid arthritis, and psoriasis. Folkman et al., J. Biol. Chem., 267: 10931-10934 (1992); Klagsbrun et al., Annu. Rev. Physiol., 53: 217-239 (1991); and Garner A., “Vascular diseases”, In: Pathobiology of Ocular Disease. A Dynamic Approach, Garner A., Klintworth G K, eds., 2nd Edition (Marcel Dekker, N Y, 1994), pp 1625-1710.

In the case of tumor growth, angiogenesis appears to be crucial for the transition from hyperplasia to neoplasia, and for providing nourishment for the growth and metastasis of the tumor. Folkman et al., Nature, 339: 58 (1989). The neovascularization allows the tumor cells to acquire a growth advantage and proliferative autonomy compared to normal cells. A tumor usually begins as a single aberrant cell which can proliferate only to a size of a few cubic millimeters due to the distance from available capillary beds, and it can stay ‘dormant’ without further growth and dissemination for a long period of time. Some tumor cells then switch to the angiogenic phenotype to activate endothelial cells, which proliferate and mature into new capillary blood vessels. These newly formed blood vessels not only allow for continued growth of the primary tumor, but also for the dissemination and recolonization of metastatic tumor cells. Accordingly, a correlation has been observed between density of microvessels in tumor sections and patient survival in breast cancer as well as in several other tumors. Weidner et al., N. Engl. J. Med, 324: 1-6 (1991); Horak et al., Lancet, 340: 1120-1124 (1992); Macchiarini et al., Lancet, 340: 145-146 (1992). The precise mechanisms that control the angiogenic switch is not well understood, but it is believed that neovascularization of tumor mass results from the net balance of a multitude of angiogenesis stimulators and inhibitors (Folkman, 1995, Nat Med 1(1):27-31).

The process of vascular development is tightly regulated. To date, a significant number of molecules, mostly secreted factors produced by surrounding cells, have been shown to regulate EC differentiation, proliferation, migration and coalescence into cord-like structures. For example, vascular endothelial growth factor (VEGF) has been identified as the key factor involved in stimulating angiogenesis and in inducing vascular permeability. Ferrara et al., Endocr. Rev., 18: 4-25 (1997). The finding that the loss of even a single VEGF allele results in embryonic lethality points to an irreplaceable role played by this factor in the development and differentiation of the vascular system. Furthermore, VEGF has been shown to be a key mediator of neovascularization associated with tumors and intraocular disorders. Ferrara et al., Endocr. Rev., supra. The VEGF mRNA is overexpressed by the majority of human tumors examined Berkman et al., J. Clin. Invest., 91: 153-159 (1993); Brown et al., Human Pathol., 26: 86-91 (1995); Brown et al., Cancer Res., 53: 4727-4735 (1993); Mattern et al., Brit. J. Cancer, 73: 931-934 (1996); Dvorak et al., Am. J. Pathol., 146: 1029-1039 (1995).

Treatment with anti-VEGF neutralizing antibodies significantly inhibited growth of several tumor cell lines, suggesting that blockade of VEGF alone may substantially suppress tumor growth through inhibition of angiogenesis (see Kim et al., Inhibition of vascular endothelial growth factor-induced angiogenesis suppresses tumour growth in vivo, Nature 362 (1993). In addition to inhibiting VEGF-A, strategies aimed at blocking VEGF receptors also result in inhibition of tumor growth ([Ellis and Hicklin, 2008], [Ferrara, 2004] and [Kerbel, 2008]). The VEGF-Trap (Aflibercept; Regeneron Inc.), a chimeric soluble receptor containing structural elements from VEGFR1 and VEGFR2 (Holash et al., 2002), inhibited tumor growth in xenograft models and is currently in clinical trials for the treatment of several tumors.

A variety of small molecule inhibitors targeting the VEGF signaling pathway has been developed. These include receptor tyrosine kinases (RTKs) inhibitors such as Bay 43-9006 (sorafenib; Nexavar®) (Kupsch et al., 2005) and SU11248 (sunitinib; Sutent®) (O'Farrell et al., 2003). Sorafenib is a raf kinase inhibitor that also inhibits VEGFR-2 and -3, PDGFR-β, Flt-3 and c-kit (Fabian et al., 2005). Sorafenib has been approved by FDA for advanced renal cell carcinoma (RCC) (Kane et al., 2006) and inoperable hepatocellular carcinoma (Lang, 2008). Similarly, sunitinib inhibits several pathways including VEGFRs, PDGFR, c-kit and Flt-3 and has shown efficacy in advanced RCC (van der Veldt et al., 2008) and in imatinib-resistant gastrointestinal stromal tumors (Smith et al., 2004).

VEGF inhibitors have demonstrated clinical efficacy and a survival advantage in patients with advanced cancer, but most patients eventually relapse. Intensive studies are underway to elucidate cellular and molecular mechanisms underlying reduced response to anti-angiogenic agents in general and VEGF blockers in particular (F. Shojaei and N. Ferrara, Refractoriness to antivascular endothelial growth factor treatment: role of myeloid cells, Cancer Res. 68 (2008), pp. 5501-5504).

Reduced response to anti-VEGF may originate from tumor and/or non-tumor (stroma) compartments. In contrast to tumor cells, stromal cells are genetically stable and do not display chromosomal abnormalities (Hughes, 2008). The stroma comprises a heterogeneous population of cells including fibroblasts, pericytes, mesenchymal stem cells and hematopoietic cells. Stromal cells support tumor growth through several possible mechanisms such as direct contribution to tumor vasculature (Santarelli et al., Incorporation of bone marrow-derived Flk-1-expressing CD34+ cells in the endothelium of tumor vessels in the mouse brain, Neurosurgery 59 (2006), pp. 374-382, release of VEGF (Liang et al., 2006) and MMP9 (Coussens et al., 2000) and Sema4D (Sierra et al., 2008) or by deflecting immune surveillance from tumor cells (Mantovani et al., 2008).

In view of the role of angiogenesis in many diseases and disorders, it is desirable to have a means of reducing or inhibiting one or more of the biological effects causing these processes. All references cited herein, including patent applications and publications, are incorporated by reference in their entirety.

### SUMMARY OF THE INVENTION

The invention provides methods of inhibiting tumor angiogenesis, of suppressing tumor growth, and of tumor treatment in a human subject having a tumor previously treated with a VEGF antagonist by administering an effective amount of an IL-17 antagonist.

In one embodiment, a method of inhibiting tumor angiogenesis, comprising administering to a human subject having a tumor previously treated with a vascular endothelial growth factor (VEGF) antagonist an effective amount of an IL-17 antagonist is contemplated. In another embodiment, in the method contemplated above, the IL-17 antagonist is an anti-IL-17 antibody or fragment thereof or an anti-IL-17 receptor antibody or a fragment thereof. In yet another embodiment, said anti-IL-17 antibody or fragment thereof specifically binds to IL-17A or IL-17F or IL-17A and IL-17F. In a further embodiment, in the method contemplated above, the tumor is refractory to treatment with said VEGF antagonist. In yet a further embodiment, the VEGF antagonist is an anti-VEGF antibody of fragment thereof. In yet another embodiment, the anti-VEGF antibody is bevacizumab comprising a variable heavy chain sequence of SEQ ID NO: 1 and a variable light chain sequence of SEQ ID NO: 2, or fragment or variant thereof. In one embodiment, the IL-17 antibody or fragment thereof is a monoclonal antibody. In another embodiment, the IL-17 antibody or fragment thereof is a human, a humanized or a chimeric antibody.

In one embodiment, in the method contemplated above, the IL-17 antagonist or IL-17 antibody or fragment thereof decreases mean vascular density in said tumor as compared to a tumor in a human subject that was not administered an effective amount of an IL-17 antagonist or IL-17 antibody or fragment thereof. In a further embodiment in the method contemplated above, said inhibition of tumor angiogenesis decreases mean vascular density in said tumor in said human subject by at least 5%, or at least 10%, or at least 15%, or at least 20%, or at least 25%, or at least 30%, or at least 35%, or at least 40%, or at least 45%, or at least 50%, or at least 55%, or at least 60%, or at least 65% or at least 70%, or at least 75%, or at least 80%, or at least 85%, or at least 90%, or at least 95% or at least 99%, as compared to a tumor in a human subject that was not administered an effective amount of an IL-17 antagonist or IL-17 antibody or fragment thereof. In yet another embodiment, in the method contemplated above, said mean vascular density is measured by taking an average area of CD31-positive cells in said tumor over a total area of cells in said tumor.

In yet another embodiment, the method further comprises administering to said human subject an anti-VEGF antibody or fragment thereof. In another embodiment of the method contemplated above, the anti-VEGF antibody is bevacizumab comprising a variable heavy chain sequence of SEQ ID NO: 1 and a variable light chain sequence of SEQ ID NO: 2, or fragment thereof. In yet another embodiment, the method further comprises subjecting said human subject to chemotherapy or radiation therapy. In yet another embodiment, the method further comprises administering an effective amount of a G-CSF antagonist. In another embodiment, said G-CSF antagonist is an anti-G-CSF antibody or fragment thereof. In yet another embodiment, said anti-G-CSF antibody or fragment thereof is a monoclonal antibody. In yet another embodiment, said anti-G-CSF antibody or fragment thereof is a human, a humanized or a chimeric antibody.

In one embodiment, in the method contemplated above, the tumor is in the colon, rectum, liver, lung, prostate, breast or ovary.

In another embodiment, the method contemplated above further comprises monitoring the efficacy of said inhibition of tumor angiogenesis by determining the number or frequency of CD11b+Gr1+ cells in a tumor sample or in a peripheral blood sample obtained from said human subject, relative to the number or frequency of a tumor sample or peripheral blood sample obtained from said human subject prior to administration of said IL-17 antagonist.

In one embodiment, a method of suppressing tumor growth, comprising administering to a human subject having a tumor previously treated with a vascular endothelial growth factor (VEGF) antagonist an effective amount of an IL-17 antagonist is contemplated. In another embodiment, in the method contemplated above, the IL-17 antagonist is an anti-IL-17 antibody or fragment thereof or an anti-IL-17 receptor antibody or a fragment thereof. In yet another embodiment, said anti-IL-17 antibody or fragment thereof specifically binds to IL-17A or IL-17F or IL-17A and IL-17F. In a further embodiment, in the method contemplated above, the tumor is refractory to treatment with said VEGF antagonist. In yet a further embodiment, the VEGF antagonist is an anti-VEGF antibody of fragment thereof. In yet another embodiment, the anti-VEGF antibody is bevacizumab comprising a variable heavy chain sequence of SEQ ID NO: 1 and a variable light chain sequence of SEQ ID NO: 2, or fragment or variant thereof. In one embodiment, the IL-17 antibody or fragment thereof is a monoclonal antibody. In another embodiment, the IL-17 antibody or fragment thereof is a human, a humanized or a chimeric antibody.

In one embodiment, in the method contemplated above, the IL-17 antagonist or IL-17 antibody or fragment thereof decreases mean vascular density in said tumor as compared to a tumor in a human subject that was not administered an effective amount of an IL-17 antagonist or IL-17 antibody or fragment thereof. In a further embodiment in the method contemplated above, said suppression of tumor growth decreases tumor volume in said human subject by at least 5%, or at least 10%, or at least 15%, or at least 20%, or at least 25%, or at least 30%, or at least 35%, or at least 40%, or at least 45%, or at least 50%, or at least 55%, or at least 60%, or at least 65% or at least 70%, or at least 75%, or at least 80%, or at least 85%, or at least 90%, or at least 95% or at least 99%, as compared to a tumor in a human subject that was not administered an effective amount of an IL-17 antagonist or IL-17 antibody or fragment thereof. In another embodiment, said decrease in tumor volume is measured by computerized axial tomography (CAT Scan), magnetic resonance imaging (MRI), positron emission tomography (PET), or single-photon emission computed tomography (SPECT).

In yet another embodiment, the method further comprises administering to said human subject an anti-VEGF antibody or fragment thereof. In another embodiment of the method contemplated above, the anti-VEGF antibody is bevacizumab or fragment thereof. In yet another embodiment, the method further comprises subjecting said human subject to chemotherapy or radiation therapy. In yet another embodiment, the method further comprises administering an effective amount of a G-CSF antagonist. In another embodiment, said G-CSF antagonist is an anti-G-CSF antibody or fragment thereof. In yet another embodiment, said anti-G-CSF antibody or fragment thereof is a monoclonal antibody. In yet another embodiment, said anti-G-CSF antibody or fragment thereof is a human, a humanized or a chimeric antibody.

In one embodiment, in the method contemplated above, the tumor is in the colon, rectum, liver, lung, prostate, breast or ovary.

In another embodiment, the method contemplated above further comprises monitoring the efficacy of said suppression of tumor growth by determining the number or frequency of CD11b+Gr1+ cells in a tumor sample or in a peripheral blood sample obtained from said human subject, relative to the number or frequency of a tumor sample or peripheral blood sample obtained from said human subject prior to administration of said IL-17 antagonist.

In one embodiment, a method of tumor treatment, comprising administering to a human subject having a tumor previously treated with a vascular endothelial growth factor (VEGF) antagonist an effective amount of an IL-17 antagonist is contemplated. In another embodiment, in the method contemplated above, the IL-17 antagonist is an anti-IL-17 antibody or fragment thereof or an anti-IL-17 receptor antibody or a fragment thereof. In yet another embodiment, said anti-IL-17 antibody or fragment thereof specifically binds to IL-17A or IL-17F or IL-17A and IL-17F. In a further embodiment, in the method contemplated above, the tumor is refractory to treatment with said VEGF antagonist. In yet a further embodiment, the VEGF antagonist is an anti-VEGF antibody of fragment thereof. In yet another embodiment, the anti-VEGF antibody is bevacizumab comprising a variable heavy chain sequence of SEQ ID NO: 1 and a variable light chain sequence of SEQ ID NO: 2, or fragment or variant thereof. In one embodiment, the IL-17 antibody or fragment thereof is a monoclonal antibody. In another embodiment, the IL-17 antibody or fragment thereof is a human, a humanized or a chimeric antibody.

In one embodiment, in the method contemplated above, the IL-17 antagonist or IL-17 antibody or fragment thereof decreases mean vascular density in said tumor as compared to a tumor in a human subject that was not administered an effective amount of an IL-17 antagonist or IL-17 antibody or fragment thereof. In a further embodiment in the method contemplated above, said tumor treatment decreases tumor volume in said human subject by at least 5%, or at least 10%, or at least 15%, or at least 20%, or at least 25%, or at least 30%, or at least 35%, or at least 40%, or at least 45%, or at least 50%, or at least 55%, or at least 60%, or at least 65% or at least 70%, or at least 75%, or at least 80%, or at least 85%, or at least 90%, or at least 95% or at least 99%, as compared to a tumor in a human subject that was not administered an effective amount of an IL-17 antagonist or IL-17 antibody or fragment thereof. In another embodiment, said decrease in tumor volume is measured by computerized axial tomography (CAT Scan), magnetic resonance imaging (MRI), positron emission tomography (PET), or single-photon emission computed tomography (SPECT).

In yet another embodiment, the method further comprises administering to said human subject an anti-VEGF antibody or fragment thereof. In another embodiment of the method contemplated above, the anti-VEGF antibody is bevacizumab comprising a variable heavy chain sequence of SEQ ID NO: 1 and a variable light chain sequence of SEQ ID NO: 2, or fragment thereof. In yet another embodiment, the method further comprises subjecting said human subject to chemotherapy or radiation therapy. In yet another embodiment, the method further comprises administering an effective amount of a G-CSF antagonist. In another embodiment, said G-CSF antagonist is an anti-G-CSF antibody or fragment thereof. In yet another embodiment, said anti-G-CSF antibody or fragment thereof is a monoclonal antibody. In yet another embodiment, said anti-G-CSF antibody or fragment thereof is a human, a humanized or a chimeric antibody.

In one embodiment, in the method contemplated above, the tumor is in the colon, rectum, liver, lung, prostate, breast or ovary.

In another embodiment, the method contemplated above further comprises monitoring the efficacy of said tumor treatment by determining the number or frequency of CD11b+Gr1+ cells in a tumor sample or in a peripheral blood sample obtained from said human subject, relative to the number or frequency of a tumor sample or peripheral blood sample obtained from said human subject prior to administration of said IL-17 antagonist.

### BRIEF DESCRIPTION OF THE DRAWINGS

FIGS. 1A-B demonstrate the secreted protein profile of anti-VEGF resistant tumor cells (EL4) versus anti-VEGF sensitive tumor cells (Tib6) in culture. FIG. 1A shows that 11-17 is the most abundant secreted factor found in anti-VEGF resistant (EL4) vs. sensitive (Tib6) tumor cells in vitro. FIG. 1B shows that IL-6 and G-CSF levels are elevated in co-cultures of anti-VEGF refractory EL4 cells and normal skin fibroblasts (NSF).

FIGS. 2A-C show GFP-labeled EL4 tumor cells were co-cultured with normal skin fibroblasts (NSF) for 72 h, followed by FACS isolation of tumor cells from fibroblasts and analysis of gene expression by qRT-PCR. FIG. 2A shows G-CSF (Csf3), FIG. 2B shows IL-6, and FIG. 2C shows IL-17 induced expression were observed in normal fibroblasts when in co-cultured with the anti-VEGF resistant cell line EL4 versus mono-cultured cells. A pre-sort analysis was performed to control for potential artifacts introduced by FACS sorting.

FIG. 3 shows IL-17 neutralization inhibits EL4 induced G-CSF expression in fibroblasts induced by EL4 tumor cells in co-culture.

FIGS. 4A and B show growth of EL4 tumors in: FIG. 4A—C57/BL6 WT and Il-17rc−/− mice treated with control antibody (anti-Ragweed, 10 mg/kg, intraperitoneally (IP), twice weekly) or anti-VEGF (10 mg/kg, IP, twice weekly); and FIG. 4B—C57/BL6 WT mice treated with control antibody (anti-Ragweed), IL-17A-neutralizing antibody, anti-VEGF or antibodies to IL-17 and VEGF in combination. Data are shown as mean±SEM. *indicates significant difference (P<0.0001) between EL4 tumors in WT and Il-17rc−/− animals treated with anti-VEGF.

FIGS. 5A-C show serum levels of FIG. 5A: mG-CSF, FIG. 5B: Bv8, and FIG. 5C: IL-17A in EL4-bearing WT and Il-17rc−/− mice treated with either a control anti-Ragweed antibody (Rag, 10 mg/kg) or anti-VEGF antibody (B20, 10 mg/kg). Data are shown as means±SD.

FIGS. 6A-B shows whole blood cells (WBC), isolated from tumor bearing mice, stained with Gr1+/CD11b+ Abs and sorted by FACS from wild-type (WT) or Il-17rc−/− mice (KO) (FIG. 6A) or naïve mice (FIG. 6B), treated with either control anti-Ragweed antibody (Rag) or anti-VEGF antibody (B20) Immune-suppressive immature myeloid cells are defined as Gr1+/CD11b+ double-positive cells.

FIGS. 7A-C show the same data as in FIG. 6, where the number of Gr1+/CD11b+ double-positive cells are quantified in FIG. 7A: blood, FIG. 7B: spleen, and FIG. 7C: tumor.**p<0.0005, *p<0.5.

FIGS. 8A-D show Gr1+ cells isolated from spleens of tumor-bearing mice cultured overnight in the absence or presence of LPS followed by qRT-PCR analysis for the expression of pro-angiogenic genes such as Bv8 (FIG. 8A) and tumor promoting genes such as S100A8 (FIG. 8B); MMP9 (FIG. 8C); and S100A9 (FIG. 8D).

FIG. 9 shows EL4 tumors derived from WT and IL-17rc KO animals, treated with control antibody (anti-Ragweed) or anti-VEGF antibody (clone B20) immunostained with anti-CD31 (endothelial cell) as shown in green, anti-Desmin (mural cell) as shown in blue, and anti-smooth muscle actin (SMA) as shown in red, where positive staining corresponds to arterioles in this tumor type.

FIG. 10 shows the same data as in FIG. 9 as a quantification of immunostaining, represented as average area of CD31-positive cells over total area of cells. Whole tumor cross-sections from at least 5 different mice/group were used for quantification. *p<0.05. Error bars are represented as ±SEM.

FIG. 11 shows that paracrine IL-17 function is required for refractoriness to anti-VEGF treatment using Tib6 cell lines, which are sensitive to VEGF treatment. Changes in tumor volume (y-axis as Rel Vol (%)) of Tib6-neo tumor cell lines (n=2) (solid line) and Tib-6 tumor lines stably expressing mIL-17A (Tib6-IL-17; n=4) (dashed line) treated with anti-VEGF are shown. Data represented as mean±SEM.

FIGS. 12A-C show growth curves of EL4 tumors in C57BL/6 WT mice (n=8) and G-CSFR−/− mice (n=8). FIG. 12A depicts treatment with anti-VEGF or control anti-Ragweed antibodies initiated 24 h after tumor inoculation. Quantification of levels of CD11b+Gr1+ cells entering the circulation (FIG. 12B), and infiltrating the tumor (FIG. 12C) in tumor bearing WT vs. G-CSFR−/− hosts was determined by flow cytometric analysis. Data represented as mean±SEM, and *denotes p<0.05 by Student's t-test.

FIGS. 13A-B show T_{H}17 cells mediate resistance to anti-VEGF treatment via recruitment and activation of CD11b+Gr1+ cells within the tumor microenvironment. FIG. 13A shows syngeneic subcutaneous Lewis lung carcinoma (LLC) tumor volumes over time in WT and Il-17rc−/− mice after administration of control (α-RW) and anti-VEGF antibodies. FIG. 13B depicts syngeneic CT-26 tumor volumes after administration of control, a neutralizing antibody to IL-17A (α-IL17A), anti-VEGF, or α-IL17A and anti-VEGF in combination. All data represented as mean±SEM, *denotes p<0.05 by two-tailed Student's t-test.

FIGS. 14A-B show the percentage of CD4+ (FIG. 14A) and CD4+IL-17A+IL-22+ producing tumor infiltrating lymphocytes (TILs) (FIG. 14B) from LLC tumors growing in WT and Il-17rc−/− (KO) after staining and quantification by flow cytometry.

FIGS. 15A-C show quantification of CD4+ and CD8+ TILs as described in Example 8 (FIG. 15A). The percentage of CD4+IL-17+IL-22+ out of the CD3+ population was quantified by flow cytometry (FIG. 15B). Tumor levels of IL-17A in WT and KO hosts following administration with α-RW and α-VEGF were measured by ELISA (FIG. 15C). All data represented as mean±SEM, *denotes p<0.05 by two-tailed Student's t-test.

FIGS. 16A-C show G-CSF levels within LLC tumors following treatment were measured by ELISA (FIG. 16A) followed by the number of tumor infiltrating CD11b+Gr1+ cells (FIG. 16B), tumor Bv8 levels measured by ELISA (FIG. 16C). All data represented as mean±SEM, *denotes p<0.05 by two-tailed Student's t-test.

FIG. 17 shows the number of tumor associated endothelial cells quantified by flow cytometry. n=5-8 per group. All data represented as mean±SEM, **denotes p<0.005 by two-tailed Student's t-test.

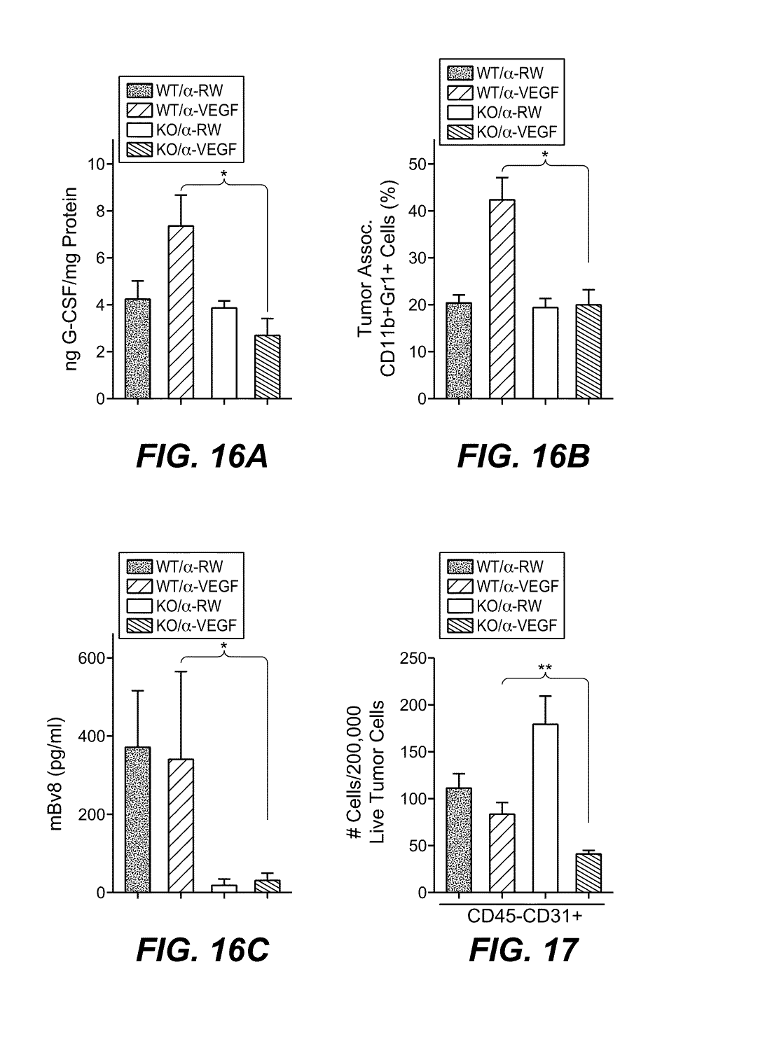

### DETAILED DESCRIPTION OF THE INVENTION

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

### A. DEFINITIONS

In general, a polypeptide “variant” (i.e. a variant of any polypeptide disclosed herein) means a biologically active polypeptide having at least about 80% amino acid sequence identity with the corresponding native sequence polypeptide. Such variants include, for instance, polypeptides wherein one or more amino acid (naturally occurring amino acid and/or a non-naturally occurring amino acid) residues are added, or deleted, at the N- and/or C-terminus of the polypeptide. Ordinarily, a variant will have at least about 80% amino acid sequence identity, or at least about 90% amino acid sequence identity, or at least about 95% or more amino acid sequence identity with the native sequence polypeptide. Variants also include polypeptide fragments (e.g., subsequences, truncations, etc.), typically biologically active, of the native sequence.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. has been filed with user documentation in the U.S. Copyright Office, Washington D.C., 20559, where it is registered under U.S. Copyright Registration No. TXU510087, and is publicly available through Genentech, Inc., South San Francisco, Calif. The ALIGN-2 program should be compiled for use on a UNIX operating system, e.g., digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The term “antagonist” when used herein refers to a molecule capable of neutralizing, blocking, inhibiting, abrogating, reducing or interfering with the activities of a protein of the invention including its binding to one or more receptors in the case of a ligand or binding to one or more ligands in case of a receptor. Antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include small molecule inhibitors of a protein of the invention, and fusions proteins, receptor molecules and derivatives which bind specifically to protein thereby sequestering its binding to its target, antagonist variants of the protein, antisense molecules directed to a protein of the invention, RNA aptamers, and ribozymes against a protein of the invention.

A “blocking” antibody or an “antagonist” antibody is one which inhibits or reduces biological activity of the antigen it binds. Certain blocking antibodies or antagonist antibodies substantially or completely inhibit the biological activity of the antigen.

The term “inhibiting tumor angiogenesis” as used herein refers to the inhibition of the ability of tumors to induce new blood-vessel formation, or to inhibit a tumor's ability to recruit existing vasculature.

The term “suppressing tumor growth” as used herein refers to a tumor that does not grow further after treatment and/or does not metastasize. Tumor growth can be suppressed when tumor angiogenesis is inhibited as described herein. As used herein, suppression of tumor growth decreases tumor volume in said human subject by at least 5%, or at least 10%, or at least 15%, or at least 20%, or at least 25%, or at least 30%, or at least 35%, or at least 40%, or at least 45%, or at least 50%, or at least 55%, or at least 60%, or at least 65% or at least 70%, or at least 75%, or at least 80%, or at least 85%, or at least 90%, or at least 95% or at least 99%, as compared to a tumor in a human subject that was not administered an effective amount of an IL-17 antagonist or IL-17 antibody or fragment thereof. Further, as used herein said decrease in tumor volume is measured by computerized axial tomography (CAT Scan), magnetic resonance imaging (MRI), positron emission tomography (PET), or single-photon emission computed tomography (SPECT), which are all well-known techniques in the art.

The term “decreasing mean vascular density in a tumor” means inhibiting or suppressing the amount or density of tumor vasculature that supports the growth of a tumor by a certain measurable amount, which is decreased by at least 5%, or at least 10%, or at least 15%, or at least 20%, or at least 25%, or at least 30%, or at least 35%, or at least 40%, or at least 45%, or at least 50%, or at least 55%, or at least 60%, or at least 65% or at least 70%, or at least 75%, or at least 80%, or at least 85%, or at least 90%, or at least 95% or at least 99%, where the decrease in vascular density is brought about by an antagonist. as compared to a tumor in a human subject that was not administered an effective amount of an IL-17 antagonist or IL-17 antibody or fragment thereof. As used herein, “mean vascular density” is measured by taking an average area of CD31-positive cells in said tumor over a total area of cells in said tumor.

The term “VEGF” as used herein refers to a native sequence vascular endothelial growth factor and varians thereof

The terms “VEGF” and “VEGF-A” are used interchangeably to refer to the native sequence 165-amino acid vascular endothelial cell growth factor and related 121-, 145-, 183-, 189-, and 206-amino acid vascular endothelial cell growth factors, as described by Leung et al. Science, 246:1306 (1989), Houck et al. Mol. Endocrin., 5:1806 (1991), and, Robinson & Stringer, Journal of Cell Science, 144(5):853-865 (2001), together with the naturally occurring allelic and processed forms thereof, as well as variants thereof. VEGF-A is part of a gene family including VEGF-B, VEGF-C, VEGF-D, VEGF-E, VEGF-F, and PlGF. VEGF-A primarily binds to two high affinity receptor tyrosine kinases, VEGFR-1 (Flt-1) and VEGFR-2 (Flk-1/KDR), the latter being the major transmitter of vascular endothelial cell mitogenic signals of VEGF-A. The term “VEGF” or “VEGF-A” also refers to VEGFs from non-human species such as mouse, rat, or primate. Sometimes the VEGF from a specific species is indicated by terms such as hVEGF for human VEGF or mVEGF for murine VEGF. The term “VEGF” is also used to refer to truncated forms or fragments of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native sequence VEGF.

A “VEGF antagonist” refers to a molecule (peptidyl or non-peptidyl) capable of neutralizing, blocking, inhibiting, abrogating, reducing or interfering with activities of a native sequence VEGF including its binding to one or more VEGF receptors. VEGF antagonists include anti-VEGF antibodies and antigen-binding fragments thereof, receptor molecules and derivatives which bind specifically to VEGF thereby sequestering its binding to one or more receptors (e.g., soluble VEGF receptor proteins, or VEGF binding fragments thereof, or chimeric VEGF receptor proteins), anti-VEGF receptor antibodies and VEGF receptor antagonists such as small molecule inhibitors of the VEGFR tyrosine kinases, and fusions proteins, e.g., VEGF-Trap (Regeneron), VEGF₁₂₁-gelonin (Peregine). VEGF antagonists also include antagonists of VEGF, antisense molecules directed to VEGF, RNA aptamers, and ribozymes against VEGF or VEGF receptors. VEGF antagonists useful in the methods of the invention further include peptidyl or non-peptidyl compounds that specifically bind VEGF, such as anti-VEGF antibodies and antigen-binding fragments thereof, polypeptides, antibody variants or fragments thereof that specifically bind to VEGF; antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers. In one embodiment, the VEGF antagonist reduces or inhibits, by at least 10%, 20%, 30%, 40%, 50%, 60%, 70%, 80%, 90% or more, the expression level or biological activity of VEGF. In another embodiment, the VEGF inhibited by the VEGF antagonist is VEGF (8-109), VEGF (1-109), or VEGF₁₆₅.

The term “anti-VEGF antibody” or “an antibody that binds to VEGF” refers to an antibody that is capable of binding to VEGF with sufficient affinity and specificity that the antibody is useful as a diagnostic and/or therapeutic agent in targeting VEGF. For example, the anti-VEGF antibody of the invention can be used as a therapeutic agent in targeting and interfering with diseases or conditions wherein the VEGF activity is involved. See, e.g., U.S. Pat. Nos. 6,582,959, 6,703,020; WO98/45332; WO 96/30046; WO94/10202, WO2005/044853; EP 0666868B1; US Patent Applications 20030206899, 20030190317, 20030203409, 20050112126, 20050186208, and 20050112126; Popkov et al., Journal of Immunological Methods 288:149-164 (2004); and WO2005012359. The antibody selected will normally have a sufficiently strong binding affinity for VEGF, for example, the antibody may bind hVEGF with a K_{d} value of between 100 nM-1 pM. Antibody affinities may be determined by a surface plasmon resonance based assay (such as the BIAcore™ assay as described in PCT Application Publication No. WO2005/012359); enzyme-linked immunoabsorbent assay (ELISA); and competition assays (e.g. RIA's), for example. The antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B, VEGF-C, VEGF-D or VEGF-E, nor other growth factors such as P1GF, PDGF or bFGF. In one embodiment, anti-VEGF antibodies include a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. (1997) Cancer Res. 57:4593-4599, including but not limited to the antibody known as “bevacizumab (BV),” also known as “rhuMAb VEGF” or AVASTN®.” Bevacizumab comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005.

In any of the methods, uses and compositions provided herein, the anti-VEGF antibody may be substituted with a VEGF specific antagonist, e.g., a VEGF receptor molecule or chimeric VEGF receptor molecule as described herein. In certain embodiments of the methods, uses and compositions provided herein, the anti-VEGF antibody is bevacizumab. The anti-VEGF antibody, or antigen-binding fragment thereof, can be a monoclonal antibody, a chimeric antibody, a fully human antibody, or a humanized antibody. Exemplary antibodies useful in the methods of the invention include bevacizumab (AVASTIN®), a G6 antibody, a B20 antibody, and fragments thereof. In certain embodiments, the anti-VEGF antibody has a heavy chain variable region comprising the following amino acid sequence:

(SEQ ID NO. 1)
EVQLVESGGG LVQPGGSLRL SCAASGYTFT NYGMNWVRQA
PGKGLEWVGW INTYTGEPTY AADFKRRFTF SLDTSKSTAY
LQMNSLRAED TAVYYCAKYP HYYGSSHWYF DVWGQGTLVT VSS

and a light chain variable region comprising the following amino acid sequence:

(SEQ ID NO. 2)
DIQMTQSPSS LSASVGDRVT ITCSASQDIS NYLNWYQQKP
GKAPKVLIYF TSSLHSGVPS RFSGSGSGTD FTLTISSLQP
EDFATYYCQQ YSTVPWTFGQ GTKVEIKR.

In some embodiments the anti-VEGF antibody comprises a CDRH1 comprising the following amino acid sequence: GYTFTNYGMN (SEQ ID NO:3), a CDRH2 comprising the following amino acid sequence: WINTYTGEPTYAADFKR (SEQ ID NO:4), a CDRH3 comprising the following amino acid sequence: YPHYYGSSHWYFDV (SEQ ID NO:5), a CDRL1 comprising the following amino acid sequence: SASQDISNYLN (SEQ ID NO:6), a CDRL2 comprising the following amino acid sequence: FTSSLHS (SEQ ID NO:7) and a CDRL3 comprising the amino acid sequence: QQYSTVPWT (SEQ ID NO:8).

Additional preferred antibodies include the G6 or B20 series antibodies (e.g., G6-23, G6-31, B20-4.1), as described in PCT Application Publication No. WO2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004).

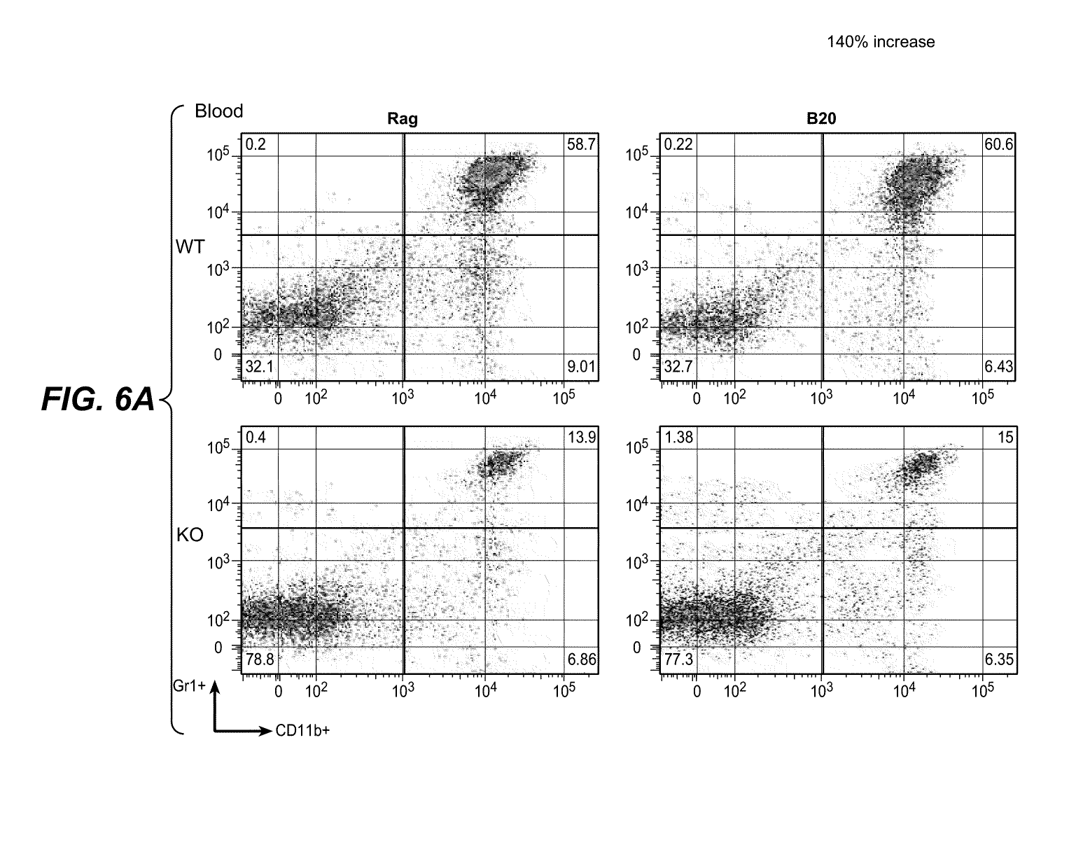

A “G6 series antibody” according to this invention, is an anti-VEGF antibody that is derived from a sequence of a G6 antibody or G6-derived antibody according to any one of FIGS. 7, 24-26, and 34-35 of PCT Publication No. WO2005/012359, the entire disclosure of which is expressly incorporated herein by reference. See also PCT Publication No. WO2005/044853, the entire disclosure of which is expressly incorporated herein by reference. In one embodiment, the G6 series antibody binds to a functional epitope on human VEGF comprising residues F17, Y21, Q22, Y25, D63, I83 and Q89.

A “B20 series antibody” according to this invention is an anti-VEGF antibody that is derived from a sequence of the B20 antibody or a B20-derived antibody according to any one of FIGS. 27-29 of PCT Publication No. WO2005/012359, the entire disclosure of which is expressly incorporated herein by reference. See also PCT Publication No. WO2005/044853, and U.S. Patent Application 60/991,302, the content of these patent applications are expressly incorporated herein by reference. In one embodiment, the B20 series antibody binds to a functional epitope on human VEGF comprising residues F17, M18, D19, Y21, Y25, Q89, I91, K101, E103, and C104.

A “hematopoietic stem/progenitor cell” or “primitive hematopoietic cell” is one which is able to differentiate to form a more committed or mature blood cell type. “Lymphoid blood cell lineages” are those hematopoietic precursor cells which are able to differentiate to form lymphocytes (B-cells or T-cells). Likewise, “lymphopoeisis” is the formation of lymphocytes. “Erythroid blood cell lineages” are those hematopoietic precursor cells which are able to differentiate to form erythrocytes (red blood cells) and “erythropoeisis” is the formation of erythrocytes.

The phrase “myeloid blood cell lineages”, for the purposes herein, encompasses all hematopoietic progenitor cells, other than lymphoid and erythroid blood cell lineages as defined above, and “myelopoiesis” involves the formation of blood cells (other than lymphocytes and erythrocytes).

A myeloid cell population can be enriched in myeloid immune cells that are Gr1+/CD11b+ (or CD11b+Gr1+) or Gr1+/Mac-1+. These cells express a marker for myeloid cells of the macrophage lineage, CD11b, and a marker for granulocytes, Gr1. A Gr1+/CD11b+ can be selected by immunoadherent panning, for example, with an antibody to Gr1+.

A “myeloid cell reduction agent” or “myeloid cell reducing agent” refers to an agent that reduces or ablates a myeloid cell population. Typically, the myeloid cell reducing agent will reduce or ablate myeloid cells, CD11b+Gr1+, monocytes, macrophages, etc. Examples of myeloid cell reducing agents include, but are not limited to, Gr1+ antagonist, CD11b antagonist, CD18 antagonist, elastase inhibitor, MCP-1 antagonist, MIP-1 alpha antagonist, etc.

The term “Gr1 antagonist” when used herein refers to a molecule which binds to Gr1 and inhibits or substantially reduces a biological activity of Gr1. Non-limiting examples of Gr1 antagonists include antibodies, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. In one embodiment of the invention, the Gr1 antagonist is an antibody, especially an anti-Gr1 antibody which binds human Gr1.

The term “CD11b antagonist” when used herein refers to a molecule which binds to CD11b and inhibits or substantially reduces a biological activity of CD11b. Normally, the antagonist will block (partially or completely) the ability of a cell (e g immature myeloid cell) expressing the CD11b subunit at its cell surface to bind to endothelium. Non-limiting examples of CD11b antagonists include antibodies, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. In one embodiment of the invention, the CD11b antagonist is an antibody, especially an anti-CD11b antibody which binds human CD11b. Exemplary CD11b antibodies include MY904 (U.S. Pat. No. 4,840,793); 1B6c (see Zhang et al., Brain Research 698:79-85 (1995)); CBRN1/5 and CBRM1/19 (WO94/08620).

A “URCGP” refers to a protein that is upregulated in CD11b+Gr+1 cells from anti-VEGF resistant tumors. URCGPs include, but are not limited to, neutropil elastase, CD14, expi, Il-13R, LDLR, TLR-1, RLF, Endo-Lip, SOCS13, FGF13, IL-4R, IL-11R, IL-1RII, IFN TM1, TNFRSF18, WNT5A, Secretory carrier membrane 1, HSP86, EGFR, EphRB2, GPCR25, HGF, Angiopoietin Like-6, Eph-RA7, Semaphorin Vlb, Neurotrophin 5, Claudin-18, MDC15, ECM and ADAMTS7B. In certain embodiment, the URCGPs refer to IL-13R, TLR-1, Endo-Lip, FGF13 and/or IL-4R.

A “DRCGP” refers to a protein that is downregulated in CD11b+Gr1+ cells from anti-VEGF resistant tumors. DRCGPs include, but are not limited to, THBS1, Crea7, Aquaporin-1, solute carrier family protein (SCF38), apolipoprotein E (APOE), fatty acid binding protein (FABP), NCAM-140, Fibronectin type III, WIP, CD74, ICAM-2, Jagged1, ltga4, ITGB7, TGF-BII-R, TGFb IEP, Smad4, BMPR1A, CD83, Dectin-1, CD48, E-selectin, IL-15, Suppressor of cytokine signaling 4, Cytor4 and CX3CR1. In certain embodiment, the DRCGPs refer to THBS1 and/or Crea7.

A “URRTP” refers to a protein that is upregulated in anti-VEGF resistant tumors. URRTPs include, but are not limited to, Notch2, DMD8, MCP-1, ITGB7, G-CSF, IL-8R, MIP2, MSCA, GM-CSF, IL-1R, Meg-SF, HSP1A, IL-1R, G-CSFR, IGF2, HSP9A, FGF18, ELM1, Ledgfa, scavenger receptor type A, Macrophage C-type lectin, Pigr3, Macrophage SRT-1, G protein-coupled receptor, ScyA7, IL-1R2, IL-1 inducible protein, IL-1beta and ILIX Precuror. In certain embodiment, the URRTPs refer to. MSCA, MIP2, IL-8R and/or G-CSF.

A “DRRTP” refers to a protein that is downregulated in anti-VEGF resistant tumors. URRTPs include, but are not limited to, IL10-R2, Erb-2.1, Caveolin3, Semcap3, INTG4, THBSP-4, ErbB3, JAM, Eng, JAM, Eng, JAM-2, Pecam1, Tlr3, TGF-B, FIZZ1, Wfs1, TP 14A, EMAP, SULF-2, Extracellular matrix 2, CTFG, TFPI, XCP2, Ramp2, ROR-alpha, Ephrin B1, SPARC-like 1, and Semaphorin A. In certain embodiments, the DRRTP refer to IL10-R2, THBSP-4, and/or JAM-2.

The term “biological sample” refers to a body sample from any animal, but preferably is from a mammal, more preferably from a human. Such samples include biological fluids such as blood, serum, plasma, bone marrow, vitreous fluid, lymph fluid, synovial fluid, follicular fluid, seminal fluid, amniotic fluid, milk, whole blood, urine, cerebro-spinal fluid, saliva, sputum, tears, perspiration, mucus, and tissue culture medium, as well as tissue extracts such as homogenized tissue, and cellular extracts. The preferred biological sample herein is serum, plasma, urine or a bone marrow sample.

The term “antibody” is used in the broadest sense and specifically covers monoclonal antibodies (including full length or intact monoclonal antibodies), polyclonal antibodies, multivalent antibodies, multispecific antibodies (e.g., bispecific antibodies) formed from at least two intact antibodies, and antibody fragments (see below) so long as they exhibit the desired biological activity.

Unless indicated otherwise, the expression “multivalent antibody” is used throughout this specification to denote an antibody comprising three or more antigen binding sites. The multivalent antibody is typically engineered to have the three or more antigen binding sites and is generally not a native sequence IgM or IgA antibody.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH—CH1-VH—CH1) which, together with complementary light chain polypeptides, form a pair of antigen binding regions (Zapata et al. Protein Eng. 8(10):1057 1062 (1995); and U.S. Pat. No. 5,641,870).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N. Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1991); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409. See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Set. USA, 1.03:3557-3562 (2006) regarding human antibodies generated via a human B-cell hybridoma technology.

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cellular toxicity.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra. A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below in Table 1.

TABLE 1
Residue Comparisons for HVRs according to Kabat, AbM and Chothia

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3 (L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

Throughout the present specification and claims, the Kabat numbering system is generally used when referring to a residue in the variable domain (approximately, residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, Md. (1991) expressly incorporated herein by reference). Unless stated otherwise herein, references to residues numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁ (including non-A and A allotypes), IgG₂, IgG₃, IgG₄, IgA₁, and IgA₂. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The “light chains” of antibodies (immunoglobulins) from any vertebrate species can be assigned to one of two clearly distinct types, called kappa (κ) and lambda (λ) based on the amino acid sequences of their constant domains.

The term “Fc region” is used to define the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. The Fc region of an immunoglobulin generally comprises two constant domains, a CH2 domain and a CH3 domain, and optionally comprises a CH4 domain.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

By “Fc region chain” herein is meant one of the two polypeptide chains of an Fc region.

The “CH2 domain” of a human IgG Fc region (also referred to as “Cg2” domain) usually extends from an amino acid residue at about position 231 to an amino acid residue at about position 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985). The CH2 domain herein may be a native sequence CH2 domain or variant CH2 domain.

The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protroberance” in one chain thereof and a corresponding introduced “cavity” in the other chain thereof; see U.S. Pat. No. 5,821,333, expressly incorporated herein by reference). Such variant CH3 domains may be used to make multispecific (e.g. bispecific) antibodies as herein described.

“Hinge region” is generally defined as stretching from about Glu216, or about Cys226, to about Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions. The hinge region herein may be a native sequence hinge region or a variant hinge region. The two polypeptide chains of a variant hinge region generally retain at least one cysteine residue per polypeptide chain, so that the two polypeptide chains of the variant hinge region can form a disulfide bond between the two chains. The preferred hinge region herein is a native sequence human hinge region, e.g. a native sequence human IgG1 hinge region.

A “functional Fc region” possesses at least one “effector function” of a native sequence Fc region. Exemplary “effector functions” include Clq binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

An “intact” antibody is one which comprises an antigen-binding variable region as well as a light chain constant domain (CO and heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

A “parent antibody” or “wild-type” antibody is an antibody comprising an amino acid sequence which lacks one or more amino acid sequence alterations compared to an antibody variant as herein disclosed. Thus, the parent antibody generally has at least one hypervariable region which differs in amino acid sequence from the amino acid sequence of the corresponding hypervariable region of an antibody variant as herein disclosed. The parent polypeptide may comprise a native sequence (i.e. a naturally occurring) antibody (including a naturally occurring allelic variant), or an antibody with pre-existing amino acid sequence modifications (such as insertions, deletions and/or other alterations) of a naturally occurring sequence. Throughout the disclosure, “wild type,” “WT,” “wt,” and “parent” or “parental” antibody are used interchangeably.

As used herein, “antibody variant” or “variant antibody” refers to an antibody which has an amino acid sequence which differs from the amino acid sequence of a parent antibody. Preferably, the antibody variant comprises a heavy chain variable domain or a light chain variable domain having an amino acid sequence which is not found in nature. Such variants necessarily have less than 100% sequence identity or similarity with the parent antibody. In a preferred embodiment, the antibody variant will have an amino acid sequence from about 75% to less than 100% amino acid sequence identity or similarity with the amino acid sequence of either the heavy or light chain variable domain of the parent antibody, more preferably from about 80% to less than 100%, more preferably from about 85% to less than 100%, more preferably from about 90% to less than 100%, and most preferably from about 95% to less than 100%. The antibody variant is generally one which comprises one or more amino acid alterations in or adjacent to one or more hypervariable regions thereof.

A “variant Fc region” comprises an amino acid sequence which differs from that of a native sequence Fc region by virtue of at least one amino acid modification. In certain embodiments, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide, e.g. from about one to about ten amino acid substitutions, and preferably from about one to about five amino acid substitutions in a native sequence Fc region or in the Fc region of the parent polypeptide. The variant Fc region herein will typically possess, e.g., at least about 80% sequence identity with a native sequence Fc region and/or with an Fc region of a parent polypeptide, or at least about 90% sequence identity therewith, or at least about 95% sequence or more identity therewith.

Antibody “effector functions” refer to those biological activities attributable to the Fc region (a native sequence Fc region or amino acid sequence variant Fc region) of an antibody, and vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells and neutrophils; with PBMCs and NK cells being generally preferred. The effector cells may be isolated from a native source thereof, e.g. from blood or PBMCs as described herein.

“Fc receptor” or “FcR” describes a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See also, e.g., Shields et al. J. Biol. Chem. 9(2):6591-6604 (2001).

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased Clq binding capability are described, e.g., in U.S. Pat. No. 6,194,551 B1 and WO 1999/51642. See also, e.g., Idusogie et al. J. Immunol. 164: 4178-4184 (2000).

An “affinity matured” antibody is one with one or more alterations in one or more CDRs thereof which result an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “flexible linker” herein refers to a peptide comprising two or more amino acid residues joined by peptide bond(s), and provides more rotational freedom for two polypeptides (such as two Fd regions) linked thereby. Such rotational freedom allows two or more antigen binding sites joined by the flexible linker to each access target antigen(s) more efficiently. Examples of suitable flexible linker peptide sequences include gly-ser, gly-ser-gly-ser (SEQ ID NO: 34), ala-ser, and gly-gly-gly-ser (SEQ ID NO: 35).

A “dimerization domain” is formed by the association of at least two amino acid residues (generally cysteine residues) or of at least two peptides or polypeptides (which may have the same, or different, amino acid sequences). The peptides or polypeptides may interact with each other through covalent and/or non-covalent association(s). Examples of dimerization domains herein include an Fc region; a hinge region; a CH3 domain; a CH4 domain; a CH1-CL pair; an “interface” with an engineered “knob” and/or “protruberance” as described in U.S. Pat. No. 5,821,333, expressly incorporated herein by reference; a leucine zipper (e.g. a jun/fos leucine zipper, see Kostelney et al., J. Immunol., 148: 1547-1553 (1992); or a yeast GCN4 leucine zipper); an isoleucine zipper; a receptor dimer pair (e.g., interleukin-8 receptor (IL-8R); and integrin heterodimers such as LFA-1 and GPIIIb/IIIa), or the dimerization region(s) thereof; dimeric ligand polypeptides (e.g. nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF); see Arakawa et al. J. Biol. Chem. 269(45): 27833-27839 (1994) and Radziejewski et al. Biochem. 32(48): 1350 (1993)), or the dimerization region(s) thereof; a pair of cysteine residues able to form a disulfide bond; a pair of peptides or polypeptides, each comprising at least one cysteine residue (e.g. from about one, two or three to about ten cysteine residues) such that disulfide bond(s) can form between the peptides or polypeptides (hereinafter “a synthetic hinge”); and antibody variable domains. The most preferred dimerization domain herein is an Fc region or a hinge region.

A “functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen. Moreover, the antigen binding affinity of each of the antigen binding sites of a multivalent antibody herein need not be quantitatively the same. For the multimeric antibodies herein, the number of functional antigen binding sites can be evaluated using ultracentrifugation analysis. According to this method of analysis, different ratios of target antigen to multimeric antibody are combined and the average molecular weight of the complexes is calculated assuming differing numbers of functional binding sites. These theoretical values are compared to the actual experimental values obtained in order to evaluate the number of functional binding sites.

An antibody having a “biological characteristic” of a designated antibody is one which possesses one or more of the biological characteristics of that antibody which distinguish it from other antibodies that bind to the same antigen.

In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

The term “epitope” is used to refer to binding sites for (monoclonal or polyclonal) antibodies on protein antigens.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease. Specifically, the treatment may directly prevent, slow down or otherwise decrease the pathology of cellular degeneration or damage, such as the pathology of a disease or conditions associated with the mobilization of myeloid cells and/or with tumor angiogenesis.

An “effective amount” of an agent, e.g., a pharmaceutical formulation, refers to an amount effective, at dosages and for periods of time necessary, to achieve the desired therapeutic or prophylactic result.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

The term “resistant tumor” refers to cancer, cancerous cells, or a tumor that does not respond completely, or loses or shows a reduced response over the course of cancer therapy to a cancer therapy comprising at least a VEGF antagonist. A resistant tumor also refers to a tumor diagnosed as resistant herein (also referred to herein as “anti-VEGF resistant tumor”). In certain embodiments, there is an increase in CD11b+Gr1+ cells in a resistant tumor compared to a tumor that is sensitive to therapy that includes at least a VEGF antagonist.

The term “anti-neoplastic composition” refers to a composition useful in treating cancer comprising at least one active therapeutic agent, e.g., “anti-cancer agent.” Examples of therapeutic agents (anti-cancer agents) include, but are limited to, e.g., chemotherapeutic agents, growth inhibitory agents, cytotoxic agents, agents used in radiation therapy, anti-angiogenesis agents, apoptotic agents, anti-tubulin agents, toxins, and other-agents to treat cancer, e.g., anti-VEGF neutralizing antibody, VEGF antagonist, anti-G-CSF antagonist, interferons, cytokines, including an IL-17 or an IL-17 receptor or an VEGF receptor antagonists (e.g., neutralizing antibodies), and other bioactive and organic chemical agents, etc. Combinations thereof are also included in the invention.

The term “cytostatic agent” refers to a compound or composition which arrests growth of a cell either in vitro or in vivo. Thus, a cytostatic agent may be one which significantly reduces the percentage of cells in S phase. Further examples of cytostatic agents include agents that block cell cycle progression by inducing G0/G1 arrest or M-phase arrest. The humanized anti-Her2 antibody trastuzumab (HERCEPTIN®) is an example of a cytostatic agent that induces G0/G1 arrest. Classical M-phase blockers include the vincas (vincristine and vinblastine), taxanes, and topoisomerase II inhibitors such as doxorubicin, epirubicin, daunorubicin, etoposide, and bleomycin. Certain agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells. The term is intended to include radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P and radioactive isotopes of Lu), chemotherapeutic agents, and toxins such as small molecule toxins or enzymatically active toxins of bacterial, fungal, plant or animal origin, including fragments and/or variants thereof.

A “growth inhibitory agent” when used herein refers to a compound or composition which inhibits growth of a cell in vitro and/or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL®, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13.

A “chemotherapeutic agent” refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factors (e.g., VEGF, VEGF-B, VEGF-C, VEGF-D, VEGF-E); placental derived growth factor (P1GF); platelet derived growth factors (PDGF, e.g., PDGFA, PDGFB, PDGFC, PDGFD); integrin; thrombopoietin (TPO); nerve growth factors such as NGF-alpha; platelet-growth factor; transforming growth factors (TGFs) such as TGF-alpha and TGF-beta; insulin-like growth factor-I and -II; erythropoietin (EPO); osteoinductive factors; interferons such as interferon-alpha, -beta and -gamma, colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20-IL-30; secretoglobin/uteroglobin; oncostatin M (OSM); a tumor necrosis factor such as TNF-alpha or TNF-beta; and other polypeptide factors including LIF and kit ligand (KL). As used herein, the term cytokine includes proteins from natural sources or from recombinant cell culture and biologically active equivalents of the native sequence cytokines.

An “angiogenic factor or agent” is a growth factor which stimulates the development of blood vessels, e.g., promotes angiogenesis, endothelial cell growth, stability of blood vessels, and/or vasculogenesis, etc. For example, angiogenic factors, include, but are not limited to, e.g., VEGF and members of the VEGF family, P1GF, PDGF family, fibroblast growth factor family (FGFs), TIE ligands (Angiopoietins), ephrins, ANGPTL3, ANGPTL4, etc. It would also include factors that accelerate wound healing, such as growth hormone, insulin-like growth factor-I (IGF-I), VIGF, epidermal growth factor (EGF), CTGF and members of its family, and TGF-α and TGF-β. See, e.g., Klagsbrun and D'Amore, Annu. Rev. Physiol., 53:217-39 (1991); Streit and Detmar, Oncogene, 22:3172-3179 (2003); Ferrara & Alitalo, Nature Medicine 5(12):1359-1364 (1999); Tonini et al., Oncogene, 22:6549-6556 (2003) (e.g., Table 1 listing angiogenic factors); and, Sato Int. J. Clin. Oncol., 8:200-206 (2003).

An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. For example, an anti-angiogenesis agent is an antibody or other antagonist to an angiogenic agent as defined above, e.g., antibodies to VEGF, antibodies to VEGF receptors, small molecules that block VEGF receptor signaling (e.g., PTK787/ZK2284, SU6668, SUTENT/SU11248 (sunitinib malate), AMG706). Anti-angiogensis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) (e.g., Table 2 listing antiangiogenic factors); and, Sato Int. J. Clin. Oncol., 8:200-206 (2003) (e.g., Table 1 lists Anti-angiogenic agents used in clinical trials).

The term “immunosuppressive agent” as used herein refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); nonsteroidal antiinflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); hydroxycloroquine; sulfasalazine; leflunomide; cytokine or cytokine receptor antibodies including anti-interferon-alpha, -beta, or -gamma antibodies, anti-tumor necrosis factor-alpha antibodies (infliximab or adalimumab), anti-TNF-alpha immunoahesin (etanercept), anti-tumor necrosis factor-beta antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 1990/08187 published Jul. 26, 1990); streptokinase; TGF-beta; streptodornase; RNA or DNA from the host; FK506; RS-61443; deoxyspergualin; rapamycin; T-cell receptor (Cohen et al., U.S. Pat. No. 5,114,721); T-cell-receptor fragments (Offner et al., Science, 251: 430-432 (1991); WO 1990/11294; Ianeway, Nature, 341: 482 (1989); and WO 1991/01133); and T-cell-receptor antibodies (EP 340,109) such as T10B9.

Administration “in combination with” one or more further therapeutic agents includes simultaneous (concurrent) and consecutive administration in any order.

“Carriers” as used herein include pharmaceutically acceptable carriers, excipients, or stabilizers and refers to an ingredient in a pharmaceutical formulation, other than an active ingredient, which is nontoxic to a subject., A pharmaceutically acceptable carrier includes, but is not limited to, a buffer, excipient, stabilizer, or preservative.

An “effective amount” of an agent, e.g., a pharmaceutical formulation, refers to an amount effective, at dosages and for periods of time necessary, to achieve the desired therapeutic or prophylactic result.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. The term “progeny” refers to any and all offspring of every generation subsequent to an originally transformed cell or cell line. Mutant progeny that have the same function or biological activity as screened for in the originally transformed cell are included. Where distinct designations are intended, it will be clear from the context.

### Compositions and Methods

The present invention is based, at least in part, on the recognition that the IL-17 pathway plays an important and potentially dominant role in the cellular and molecular events in the tumor microenvironment leading to resistance of tumors to treatment including the administration of at least one VEGF antagonists, such as an anti-VEGF antibody. IL-17 signaling from tumors to the host stromal cells can regulate levels of pro-inflammatory and/or pro-angiogenic cytokines such as G-CSF and Bv8.

Recent studies have directly implicated CD11b+Gr1+ myeloid cells in mediating refractoriness to anti-VEGF therapy. (Shojaei, F., et al., Nature Biotechnol 25:911-20 (2007)). It has been shown that the mobilization and activation of CD11b+Gr1+ myeloid cells can result in the resistance to anti-VEGF treatment (Shojaei et al 2009). It has also been shown that bone marrow-derived CD11b+Gr1+ myeloid cells isolated from tumor-bearing mice can confer resistance in tumors to anti-VEGF treatment and conditioned media from anti-VEGF-resistant (but not anti-VEGF-sensitive tumors) stimulated migration of CD11b+Gr1+ cells.

The experimental data disclosed herein demonstrate that IL-17 can regulates the mobilization and tumor infiltration of CD11b+Gr1+ immune-suppressive immature myeloid cells from the bone marrow during tumor development, and thus can locally promote tumor angiogenesis. Accordingly, IL-17 is a promising target for the treatment of tumors resistant to treatment with VEGF antagonists.

### A. MAKING ANTI-IL-17 ANTIBODIES

The antibodies identified by the binding and activity assays of the present invention can be produced by methods known in the art, including techniques of recombinant DNA technology.

i) Antigen Preparation

Soluble antigens or fragments thereof, optionally conjugated to other molecules, can be used as immunogens for generating antibodies. For transmembrane molecules, such as receptors, fragments of these (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

(ii) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCI₂, or R₁N═C═NR, where R and R₁ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(iii) Monoclonal Antibodies

Monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al, Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In a further embodiment, antibodies or antibody fragments can be isolated from antibody phage libraries generated using the techniques described in McCafferty et al., Nature, 348:552-554 (1990).

Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iv) Humanized and Human Antibodies

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad Sci. USA, 89:4285 (1992); Presta et al., J. Immnol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J.sub.H) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992). Human antibodies can also be derived from phage-display libraries (Hoogenboom et al, J. Mol. Biol., 227:381 (1991); Marks et al, J. MoL Biol., 222:581-597 (1991); Vaughan et al. Nature Biotech 14:309 (1996)). Generation of human antibodies from antibody phage display libraries is further described below.

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). In another embodiment as described in the example below, the F(ab′)₂ is formed using the leucine zipper GCN4 to promote assembly of the F(ab′)₂ molecule. According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

(vi) Multispecific Antibodies

Multispecific antibodies have binding specificities for at least two different epitopes, where the epitopes are usually from different antigens. While such molecules normally will only bind two different epitopes (i.e. bispecific antibodies, BsAbs), antibodies with additional specificities such as trispecific antibodies are encompassed by this expression when used herein. Examples of BsAbs include those with one arm directed against IL-17 and another arm directed against VEGF or G-CSF.

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991). According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in WO96/27011, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′-SH fragments can also be directly recovered from E. coli, and can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Nati. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al, J. Immunol, 152:5368 (1994).

Antibodies with more than two valencies are contemplated. For example, trispecific antibodies can be prepared. Tuft et al. J. Immunol. 147: 60 (1991).

(vii) Effector Function Engineering

It may be desirable to modify the antibody of the invention with respect to effector function, so as to enhance the effectiveness of the antibody. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctonal cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al Anti-Cancer Drug Design 3:219-230 (1989).

(viii) Antibody-Salvage Receptor Binding Epitope Fusions.

In certain embodiments of the invention, it may be desirable to use an antibody fragment, rather than an intact antibody, to increase tumor penetration, for example. In this case, it may be desirable to modify the antibody fragment in order to increase its serum half life. This may be achieved, for example, by incorporation of a salvage receptor binding epitope into the antibody fragment (e.g. by mutation of the appropriate region in the antibody fragment or by incorporating the epitope into a peptide tag that is then fused to the antibody fragment at either end or in the middle, e.g., by DNA or peptide synthesis).

The salvage receptor binding epitope preferably constitutes a region wherein any one or more amino acid residues from one or two loops of a Fc domain are transferred to an analogous position of the antibody fragment. Even more preferably, three or more residues from one or two loops of the Fc domain are transferred. Still more preferred, the epitope is taken from the CH2 domain of the Fc region (e.g., of an IgG) and transferred to the CH1, CH3, or V.sub.H region, or more than one such region, of the antibody. Alternatively, the epitope is taken from the CH2 domain of the Fc region and transferred to the CL region or VL region, or both, of the antibody fragment.

(ix) Other Covalent Modifications of Antibodies

Covalent modifications of antibodies are included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues. Examples of covalent modifications are described in U.S. Pat. No. 5,534,615, specifically incorporated herein by reference. A preferred type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

The invention also pertains to immunoconjugates comprising the antibody described herein conjugated to a cytotoxic agent such as a chemotherapeutic agent, toxin (e.g. an enzymatically active toxin of bacterial, fungal, plant or animal origin, or fragments thereof), or a radioactive isotope (i.e., a radioconjugate). A variety of radionuclides are available for the production of radioconjugate antibodies. Examples include, but are not limited to, e.g., ²¹²Bi, ¹³¹I, ¹³¹In, ⁹⁰Y and ¹⁸⁶Re.

Chemotherapeutic agents useful in the generation of such immunoconjugates have been described above. For example, BCNU, streptozoicin, vincristine, 5-fluorouracil, the family of agents known collectively LL-E33288 complex described in U.S. Pat. Nos. 5,053,394, 5,770,710, esperamicins (U.S. Pat. No. 5,877,296), etc. (see also the definition of chemotherapeutic agents herein) can be conjugated to antibodies of the invention or fragments thereof

For selective destruction of the tumor, the antibody may comprise a highly radioactive atom. A variety of radioactive isotopes are available for the production of radioconjugated antibodies or fragments thereof. Examples include, but are not limited to, e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb, ¹¹¹In, radioactive isotopes of Lu, etc. When the conjugate is used for diagnosis, it may comprise a radioactive atom for scintigraphic studies, for example ^{99m}tc or ¹²³I, or a spin label for nuclear magnetic resonance (NMR) imaging (also known as magnetic resonance imaging, MRI), such as iodine-123, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as ^{99m}tc or ¹²³I, ¹⁸⁶Re, ¹⁸⁸Re and ¹¹¹In can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. See, e.g., Monoclonal Antibodies in Immunoscintigraphy (Chatal, CRC Press 1989) which describes other methods in detail.

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, sapaonaria officinalis inhibitor, gelonin, mitogellin, restrictocin, phenomycin, neomycin, and the tricothecenes. See, e.g., WO 93/21232 published Oct. 28, 1993.

Conjugates of the antibody and cytotoxic agent are made using a variety of bifunctional protein coupling agents such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

Alternatively, a fusion protein comprising the anti-IL-17 antibody, and/or an anti-VEGF antibody and/or an anti-GCSF antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In certain embodiments, the antibody is conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). In certain embodiments, an immunoconjugate is formed between an antibody and a compound with nucleolytic activity (e.g., a ribonuclease or a DNA endonuclease such as a deoxyribonuclease; Dnase).

The invention provides an antibody of the invention, which is conjugated to one or more maytansinoid molecules. Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

An antibody of the invention can be conjugated to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. In one embodiment, maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, and Chari et al., Cancer Research 52:127-131 (1992). The linking groups include disulfide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Typical coupling agents include N-succinimidyl-3-(2-pyridyldithio) propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. The linkage is formed at the C-3 position of maytansinol or a maytansinol analogue.

Another immunoconjugate of interest comprises an antibody of the invention conjugated to one or more calicheamicin molecules. The calicheamicin family of antibiotics is capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ^{I}₁ (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

(x) Generation of Antibodies from Synthetic Antibody Phage Libraries

In an embodiment, the antibodies described herein are generated and selected using a unique phage display approach. The approach involves generation of synthetic antibody phage libraries based on single framework template, design of sufficient diversities within variable domains, display of polypeptides having the diversified variable domains, selection of candidate antibodies with high affinity to target the antigen, and isolation of the selected antibodies.

Details of the phage display methods can be found, for example, WO03/102157 published Dec. 11, 2003, the entire disclosure of which is expressly incorporated herein by reference.

In one aspect, the antibody libraries used can be generated by mutating the solvent accessible and/or highly diverse positions in at least one CDR of an antibody variable domain. Some or all of the CDRs can be mutated using the methods provided herein. In some embodiments, it may be preferable to generate diverse antibody libraries by mutating positions in CDRH1, CDRH2 and CDRH3 to form a single library or by mutating positions in CDRL3 and CDRH3 to form a single library or by mutating positions in CDRL3 and CDRH1, CDRH2 and CDRH3 to form a single library.

A library of antibody variable domains can be generated, for example, having mutations in the solvent accessible and/or highly diverse positions of CDRH1, CDRH2 and CDRH3. Another library can be generated having mutations in CDRL1, CDRL2 and CDRL3. These libraries can also be used in conjunction with each other to generate binders of desired affinities. For example, after one or more rounds of selection of heavy chain libraries for binding to a target antigen, a light chain library can be replaced into the population of heavy chain binders for further rounds of selection to increase the affinity of the binders.

Preferably, a library is created by substitution of original amino acids with variant amino acids in the CDRH3 region of the variable region of the heavy chain sequence. The resulting library can contain a plurality of antibody sequences, wherein the sequence diversity is primarily in the CDRH3 region of the heavy chain sequence.

In one aspect, the library is created by substitution of at least residues 95-100a of the heavy chain with amino acids encoded by the DVK codon set, wherein the DVK codon set is used to encode a set of variant amino acids for every one of these positions. An example of an oligonucleotide set that is useful for creating these substitutions comprises the sequence (DVK)₇. In some embodiments, a library is created by substitution of residues 95-100a with amino acids encoded by both DVK and NNK codon sets. An example of an oligonucleotide set that is useful for creating these substitutions comprises the sequence (DVK)₆ (NNK). In another embodiment, a library is created by substitution of at least residues 95-100a with amino acids encoded by both DVK and NNK codon sets. An example of an oligonucleotide set that is useful for creating these substitutions comprises the sequence (DVK)₅ (NNK). Another example of an oligonucleotide set that is useful for creating these substitutions comprises the sequence (NNK)₆. Other examples of suitable oligonucleotide sequences can be determined by one skilled in the art according to the criteria described herein.

In another embodiment, different CDRH3 designs are utilized to isolate high affinity binders and to isolate binders for a variety of epitopes. The range of lengths of CDRH3 generated in this library is 11 to 13 amino acids, although lengths different from this can also be generated. H3 diversity can be expanded by using NNK, DVK and NVK codon sets, as well as more limited diversity at N and/or C-terminal

Diversity can also be generated in CDRH1 and CDRH2. The designs of CDR-H1 and H2 diversities follow the strategy of targeting to mimic natural antibodies repertoire as described with modification that focus the diversity more closely matched to the natural diversity than previous design.

For diversity in CDRH3, multiple libraries can be constructed separately with different lengths of H3 and then combined to select for binders to target antigens. The multiple libraries can be pooled and sorted using solid support selection and solution sorting methods as described previously and herein below. Multiple sorting satrategies may be employed. For example, one variation involves sorting on target bound to a solid, followed by sorting for a tag that may be present on the fusion polypeptide (eg. anti-gD tag) and followed by another sort on target bound to solid. Alternatively, the libraries can be sorted first on target bound to a solid surface, the eluted binders are then sorted using solution phase binding with decreasing concentrations of target antigen. Utilizing combinations of different sorting methods provides for minimization of selection of only highly expressed sequences and provides for selection of a number of different high affinity clones.

High affinity binders for the target antigen can be isolated from the libraries. Limiting diversity in the H1/H2 region decreases degeneracy about 10⁴ to 10⁵ fold and allowing more H3 diversity provides for more high affinity binders. Utilizing libraries with different types of diversity in CDRH3 (eg. utilizing DVK or NVT) provides for isolation of binders that may bind to different epitopes of a target antigen.

Of the binders isolated from the pooled libraries as described above, it has been discovered that affinity may be further improved by providing limited diversity in the light chain. Light chain diversity is generated in this embodiment as follows in CDRL1: amino acid position 28 is encoded by RDT; amino acid position 29 is encoded by RKT; amino acid position 30 is encoded by RVW; amino acid position 31 is encoded by ANW; amino acid position 32 is encoded by THT; optionally, amino acid position 33 is encoded by CTG; in CDRL2: amino acid position 50 is encoded by KBG; amino acid position 53 is encoded by AVC; and optionally, amino acid position 55 is encoded by GMA; in CDRL3: amino acid position 91 is encoded by TMT or SRT or both; amino acid position 92 is encoded by DMC; amino acid position 93 is encoded by RVT; amino acid position 94 is encoded by NHT; and amino acid position 96 is encoded by TWT or YKG or both.

In another embodiment, a library or libraries with diversity in CDRH1, CDRH2 and CDRH3 regions is generated. In this embodiment, diversity in CDRH3 is generated using a variety of lengths of H3 regions and using primarily codon sets XYZ and NNK or NNS. Libraries can be formed using individual oligonucleotides and pooled or oligonucleotides can be pooled to form a subset of libraries. The libraries of this embodiment can be sorted against target bound to solid. Clones isolated from multiple sorts can be screened for specificity and affinity using ELISA assays. For specificity, the clones can be screened against the desired target antigens as well as other nontarget antigens. Those binders to the target antigen can then be screened for affinity in solution binding competition ELISA assay or spot competition assay. High affinity binders can be isolated from the library utilizing XYZ codon sets prepared as described above. These binders can be readily produced as antibodies or antigen binding fragments in high yield in cell culture.

In some embodiments, it may be desirable to generate libraries with a greater diversity in lengths of CDRH3 region. For example, it may be desirable to generate libraries with CDRH3 regions ranging from about 7 to 19 amino acids.

High affinity binders isolated from the libraries of these embodiments are readily produced in bacterial and eukaryotic cell culture in high yield. The vectors can be designed to readily remove sequences such as gD tags, viral coat protein component sequence, and/or to add in constant region sequences to provide for production of full length antibodies or antigen binding fragments in high yield.

A library with mutations in CDRH3 can be combined with a library containing variant versions of other CDRs, for example CDRL1, CDRL2, CDRL3, CDRH1 and/or CDRH2. Thus, for example, in one embodiment, a CDRH3 library is combined with a CDRL3 library created in the context of the humanized 4D5 antibody sequence with variant amino acids at positions 28, 29, 30, 31, and/or 32 using predetermined codon sets. In another embodiment, a library with mutations to the CDRH3 can be combined with a library comprising variant CDRH1 and/or CDRH2 heavy chain variable domains. In one embodiment, the CDRH1 library is created with the humanized antibody 4D5 sequence with variant amino acids at positions 28, 30, 31, 32 and 33. A CDRH2 library may be created with the sequence of humanized antibody 4D5 with variant amino acids at positions 50, 52, 53, 54, 56 and 58 using the predetermined codon sets.

(xi) Antibody Variants

The novel antibodies generated from phage libraries can be further modified to generate antibody mutants with improved physical, chemical and or biological properties over the parent antibody. Where the assay used is a biological activity assay, the antibody mutant preferably has a biological activity in the assay of choice which is at least about 10 fold better, preferably at least about 20 fold better, more preferably at least about 50 fold better, and sometimes at least about 100 fold or 200 fold better, than the biological activity of the parent antibody in that assay. For example, an anti-IL-17 antibody mutant preferably has a binding affinity for IL-17 which is at least about 10 fold stronger, preferably at least about 20 fold stronger, more preferably at least about 50 fold stronger, and sometimes at least about 100 fold or 200 fold stronger, than the binding affinity of the parent antibody.

To generate the antibody mutant, one or more amino acid alterations (e.g. substitutions) are introduced in one or more of the hypervariable regions of the parent antibody. Alternatively, or in addition, one or more alterations (e.g. substitutions) of framework region residues may be introduced in the parent antibody where these result in an improvement in the binding affinity of the antibody mutant for the antigen from the second mammalian species. Examples of framework region residues to modify include those which non-covalently bind antigen directly (Amit et al. (1986) Science 233:747-753); interact with/effect the conformation of a CDR (Chothia et al. (1987) J. Mol. Biol. 196:901-917); and/or participate in the V_{L}-V_{H} interface (EP 239 400B1). In certain embodiments, modification of one or more of such framework region residues results in an enhancement of the binding affinity of the antibody for the antigen from the second mammalian species. For example, from about one to about five framework residues may be altered in this embodiment of the invention. Sometimes, this may be sufficient to yield an antibody mutant suitable for use in preclinical trials, even where none of the hypervariable region residues have been altered. Normally, however, the antibody mutant will comprise additional hypervariable region alteration(s).

The hypervariable region residues which are altered may be changed randomly, especially where the starting binding affinity of the parent antibody is such that such randomly produced antibody mutants can be readily screened.

One useful procedure for generating such antibody mutants is called “alanine scanning mutagenesis” (Cunningham and Wells (1989) Science 244:1081-1085). Here, one or more of the hypervariable region residue(s) are replaced by alanine or polyalanine residue(s) to affect the interaction of the amino acids with the antigen from the second mammalian species. Those hypervariable region residue(s) demonstrating functional sensitivity to the substitutions then are refined by introducing further or other mutations at or for the sites of substitution. Thus, while the site for introducing an amino acid sequence variation is predetermined, the nature of the mutation per se need not be predetermined. The ala-mutants produced this way are screened for their biological activity as described herein.

Normally one would start with a conservative substitution such as those shown below under the heading of “preferred substitutions”. If such substitutions result in a change in biological activity (e.g. binding affinity), then more substantial changes, denominated “exemplary substitutions” in the following Table 2, or as further described below in reference to amino acid classes, are introduced and the products screened.

TABLE 2
Preferred Amino Acid Substitutions

| | | Preferred |
|---|---|---|
| Original Residue | Exemplary Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Even more substantial modifications in the antibodies' biological properties are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr, asn, gln;
(3) acidic: asp, glu;
(4) basic: his, lys, arg;
(5) residues that influence chain orientation: gly, pro; and
(6) aromatic: trp, tyr, phe.

Non-conservative substitutions will entail exchanging a member of one of these classes for another class.

In another embodiment, the sites selected for modification are affinity matured using phage display (see above).

Nucleic acid molecules encoding amino acid sequence mutants are prepared by a variety of methods known in the art. These methods include, but are not limited to, oligonucleotide-mediated (or site-directed) mutagenesis, PCR mutagenesis, and cassette mutagenesis of an earlier prepared mutant or a non-mutant version of the parent antibody. The preferred method for making mutants is site directed mutagenesis (see, e.g., Kunkel (1985) Proc. Natl. Acad. Sci. USA 82:488).

In certain embodiments, the antibody mutant will only have a single hypervariable region residue substituted. In other embodiments, two or more of the hypervariable region residues of the parent antibody will have been substituted, e.g. from about two to about ten hypervariable region substitutions.

Ordinarily, the antibody mutant with improved biological properties will have an amino acid sequence having at least 75% amino acid sequence identity or similarity with the amino acid sequence of either the heavy or light chain variable domain of the parent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e same residue) or similar (i e amino acid residue from the same group based on common side-chain properties, see above) with the parent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

Following production of the antibody mutant, the biological activity of that molecule relative to the parent antibody is determined. As noted above, this may involve determining the binding affinity and/or other biological activities of the antibody. In a preferred embodiment of the invention, a panel of antibody mutants is prepared and screened for binding affinity for the antigen or a fragment thereof. One or more of the antibody mutants selected from this initial screen are optionally subjected to one or more further biological activity assays to confirm that the antibody mutant(s) with enhanced binding affinity are indeed useful, e.g. for preclinical studies.

The antibody mutant(s) so selected may be subjected to further modifications, oftentimes depending on the intended use of the antibody. Such modifications may involve further alteration of the amino acid sequence, fusion to heterologous polypeptide(s) and/or covalent modifications such as those elaborated below. With respect to amino acid sequence alterations, exemplary modifications are elaborated above. For example, any cysteine residue not involved in maintaining the proper conformation of the antibody mutant also may be substituted, generally with serine, to improve the oxidative stability of the molecule and prevent aberrant cross linking. Conversely, cysteine bond(s) may be added to the antibody to improve its stability (particularly where the antibody is an antibody fragment such as an Fv fragment). Another type of amino acid mutant has an altered glycosylation pattern. This may be achieved by deleting one or more carbohydrate moieties found in the antibody, and/or adding one or more glycosylation sites that are not present in the antibody. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) on antigen-binding molecules with modified glycosylation.

The preferred glycosylation variant herein comprises an Fc region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)).

(xii) Recombinant Production of Antibodies

For recombinant production of an antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence (e.g. as described in U.S. Pat. No. 5,534,615, specifically incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serrafia, e.g, Serratia marcescans, and Shigeila, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X 1776 (ATCC 31,537), and E coil W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al, J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce the antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed cells, is removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ, γ2, or γ4 heavy chains (Lindmark et al., J. Immunol. Meth. 62:1-13 (1983)). Protein G is recommended for all mouse isotypes and for human γ3 (Guss et al., EMBO J. 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH 3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipiation are also available depending on the antibody to be recovered.

### B. USES OF IL-17 ANTAGONISTS

The IL-17 antagonists of the present invention can be used, alone or in combination with other therapeutic agent(s) for the inhibition of tumor angiogenesis.

Primary targets for the treatment methods of the present invention are tumors that have shown or are known to be resistant to treatment with VEGF antagonists, in particular anti-VEGF antibodies.

Examples of diseases and disorders to be treated by the methods of the present invention include neoplastic disorders, such as those described herein under the terms “cancer” and “cancerous.” Non-neoplastic conditions that are amenable to treatment with antagonists of the invention include, but are not limited to, e.g., undesired or aberrant hypertrophy, arthritis, rheumatoid arthritis (RA), psoriasis, psoriatic plaques, sarcoidosis, atherosclerosis, atherosclerotic plaques, edema from myocardial infarction, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertropic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), obesity, adipose tissue mass growth, hemophilic joints, hypertrophic scars, inhibition of hair growth, Osler-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

The invention provides combined therapies in which a IL-17 antagonist of the present invention is administered in combination with another therapy. Combination treatment specifically includes the administration of a IL-17 antagonist herein in combination with a VEGF antagonist, such as an anti-VEGF antibody. Alternatively, combination treatment specifically includes the administration of a IL-17 antagonist herein in combination with a G-CSF antagonist, such as an anti-G-CSF antibody. In addition, or alternatively, the IL-17 antagonists herein can be administered in combination with one or more further agents, e.g., myeloid cell reduction agent, anti-cancer agents or therapeutics, chemotherapy and/or radiation therapy, anti-angiogenesis agents, or an anti-neovascularization therapeutics to treat various neoplastic or non-neoplastic conditions, such as inflammatory cell-dependent angiogenesis or tumorigenesis.

In one embodiment, the neoplastic or non-neoplastic condition is characterized by pathological disorder associated with aberrant or undesired angiogenesis that is resistant to VEGF antagonist treatment. The antagonists of the invention can be administered serially or in combination with another agent that is effective for those purposes, either in the same composition or as separate compositions. Alternatively, or additionally, multiple antagonists, agents and/or agonists of the invention can be administered.

The administration of the antagonist and/or agents can be done simultaneously, e.g., as a single composition or as two or more distinct compositions using the same or different administration routes. Alternatively, or additionally, the administration can be done sequentially, in any order. In certain embodiments, intervals ranging from minutes to days, to weeks to months, can be present between the administrations of the two or more compositions. For example, the IL-17 antagonist may be administered first, followed by a different antagonist or agent, e.g., a VEGF and/or a G-CSF antagonist. However, simultaneous administration or administration of the different antagonist or agent of the invention first is also contemplated.

The effective amounts of therapeutic agents administered will be at the physician's or veterinarian's discretion. Dosage administration and adjustment is done to achieve maximal management of the conditions to be treated. The dose will additionally depend on such factors as the type of therapeutic agent to be used and the specific patient being treated. Suitable dosages for the IL-17 antagonist are those presently used and can be lowered due to the combined action (synergy) of the Il-17 antagonist and another antagonist of the invention, such as, for example, a VEGF and/or G-CSF antagonist. In certain embodiments, the combination of the inhibitors potentiates the efficacy of a single inhibitor. The term “potentiate” refers to an improvement in the efficacy of a therapeutic agent at its common or approved dose. See also the section entitled Pharmaceutical Compositions herein.

Anti-angiogenic therapy in relationship to cancer is a cancer treatment strategy aimed at inhibiting the development of tumor blood vessels required for providing nutrients to support tumor growth. Because angiogenesis is involved in both primary tumor growth and metastasis, the antiangiogenic treatment provided by the invention is capable of inhibiting the neoplastic growth of tumor at the primary site as well as preventing metastasis of tumors at the secondary sites, therefore allowing attack of the tumors by other therapeutics. In one embodiment of the invention, anti-cancer agent or therapeutic is an anti-angiogenic agent. In another embodiment, anti-cancer agent is a chemotherapeutic agent.

Many anti-angiogenic agents have been identified and are known in the arts, including those listed herein, e.g., listed under Definitions, and by, e.g., Carmeliet and Jain, Nature 407:249-257 (2000); Ferrara et al., Nature Reviews:Drug Discovery, 3:391-400 (2004); and Sato Int. J. Clin. Oncol., 8:200-206 (2003). See also, US Patent Application US20030055006. In one embodiment, an IL-17 antagonist of the invention is used in combination with an anti-VEGF neutralizing antibody (or fragment) and/or another VEGF antagonist or a VEGF receptor antagonist including, but not limited to, for example, soluble VEGF receptor (e.g., VEGFR-1, VEGFR-2, VEGFR-3, neuropillins (e.g., NRP1, NRP2)) fragments, aptamers capable of blocking VEGF or VEGFR, neutralizing anti-VEGFR antibodies, low molecule weight inhibitors of VEGFR tyrosine kinases (RTK), antisense strategies for VEGF, ribozymes against VEGF or VEGF receptors, antagonist variants of VEGF; and any combinations thereof. Alternatively, or additionally, two or more angiogenesis inhibitors may optionally be co-administered to the patient in addition to VEGF antagonist and other agent of the invention. In certain embodiment, one or more additional therapeutic agents, e.g., anti-cancer agents, can be administered in combination with agent of the invention, the VEGF antagonist, and/or an anti-angiogenesis agent.

In certain aspects of the invention, other therapeutic agents useful for combination tumor therapy with the IL-17 antagonists of the invention include other cancer therapies, (e.g., surgery, radiological treatments (e.g., involving irradiation or administration of radioactive substances), chemotherapy, treatment with anti-cancer agents listed herein and known in the art, or combinations thereof). Alternatively, or additionally, two or more antibodies binding the same or two or more different antigens disclosed herein can be co-administered to the patient. Sometimes, it may be beneficial to also administer one or more cytokines to the patient, such as, for example, a G-CSF antibody.

In certain aspects, the invention provides a method of blocking or reducing resistant tumor growth or growth of a cancer cell, by administering effective amounts of an antagonist of IL-17 and one or more chemotherapeutic agents to a patient susceptible to, or diagnosed with, cancer. A variety of chemotherapeutic agents may be used in the combined treatment methods of the invention. An exemplary and non-limiting list of chemotherapeutic agents contemplated is provided herein under “Definition.”

As will be understood by those of ordinary skill in the art, the appropriate doses of chemotherapeutic agents will be generally around those already employed in clinical therapies wherein the chemotherapeutics are administered alone or in combination with other chemotherapeutics. Variation in dosage will likely occur depending on the condition being treated. The physician administering treatment will be able to determine the appropriate dose for the individual subject.

The invention also provides methods and compositions for inhibiting or preventing relapse tumor growth or relapse cancer cell growth. Relapse tumor growth or relapse cancer cell growth is used to describe a condition in which patients undergoing or treated with one or more currently available therapies (e.g., cancer therapies, such as chemotherapy, radiation therapy, surgery, hormonal therapy and/or biological therapy/immunotherapy, anti-VEGF antibody therapy, particularly a standard therapeutic regimen for the particular cancer) is not clinically adequate to treat the patients or the patients are no longer receiving any beneficial effect from the therapy such that these patients need additional effective therapy. As used herein, the phrase can also refer to a condition of the “non-responsive/refractory” patient, e.g., which describe patients who respond to therapy yet suffer from side effects, develop resistance, do not respond to the therapy, do not respond satisfactorily to the therapy, etc. In various embodiments, a cancer is relapse tumor growth or relapse cancer cell growth where the number of cancer cells has not been significantly reduced, or has increased, or tumor size has not been significantly reduced, or has increased, or fails any further reduction in size or in number of cancer cells. The determination of whether the cancer cells are relapse tumor growth or relapse cancer cell growth can be made either in vivo or in vitro by any method known in the art for assaying the effectiveness of treatment on cancer cells, using the art-accepted meanings of “relapse” or “refractory” or “non-responsive” in such a context. A tumor resistant to anti-VEGF treatment is an example of a relapse tumor growth.

The invention provides methods of blocking or reducing relapse tumor growth or relapse cancer cell growth in a subject by administering one or more antagonists of the invention to block or reduce the relapse tumor growth or relapse cancer cell growth in subject. In certain embodiments, the IL-17 antagonist can be administered subsequent to the cancer therapeutic. In certain embodiments, the IL-17 antagonists of the invention are administered simultaneously with cancer therapy, e.g., chemotherapy. Alternatively, or additionally, the IL-17 antagonist therapy alternates with another cancer therapy, which can be performed in any order. The invention also encompasses methods for administering one or more inhibitory antibodies to prevent the onset or recurrence of cancer in patients predisposed to having cancer. Generally, the subject was or is concurrently undergoing cancer therapy. In one embodiment, the cancer therapy is a combination treatment with an anti-angiogenesis agent, e.g., a VEGF antagonist. The anti-angiogenesis agent includes those known in the art and those found under the Definitions herein. In one embodiment, the anti-angiogenesis agent is an anti-VEGF neutralizing antibody or fragment thereof (e.g., humanized A4.6.1, AVASTIN® (Genentech, South San Francisco, Calif.), Y0317, M4, G6, B20, 2C3, etc.). See, e.g., U.S. Pat. Nos. 6,582,959, 6,884,879, 6,703,020; WO98/45332; WO 96/30046; WO94/10202; EP 0666868B1; US Patent Applications 20030206899, 20030190317, 20030203409, and 20050112126; Popkov et al., Journal of Immunological Methods 288:149-164 (2004); and, WO2005012359. Additional agents can be administered in combination with an IL-17 antagonist for blocking or reducing relapse tumor growth or relapse cancer cell growth, e.g., see section entitled Combination Therapies herein.

In one embodiment, the IL-17 antagonists of the invention, can be administered in combination with one or more myeloid cell reduction agents, including, but not limited to therapeutics that reduce expression of Gr1, neutrophil elastase, MCP-1, MIP-1 alpha, URCGPs or URRTPs. Myeloid cell reduction agents for use in combination with the IL-17 antagonists of the present invention specifically include Gr1 antagonists, Cd11B antagonists, CD18 antagonists, elastase inhibitors, MCP-1 antagonists, MIP-1 alpha antagonist, clodronate, alone or in any combination.

In addition, the IL-17 antagonists of the present invention can be administered in combination with hormonal, radiation and chemotherapeutic agents thereby resensitizing the cancer cells to one or more of these agents, which can then be administered (or continue to be administered) to treat or manage cancer, including to prevent metastasis.

Tumor sensitivity to treatment with a IL-17 antagonist can be assessed by providing one or more test cell populations from the subject that includes cells capable of expressing one or more nucleic acid sequences homologous to nucleic acid encoding a URCGP, DRCGP, URRTP or DRRTP. Expression of the sequences is compared to a reference cell population. Any reference cell population can be used, as long as the IL-17 antagonist sensitivity status of the cells in the reference cell population is known. Comparison can be performed on test and reference samples measured concurrently or at temporally distinct times. An example of the latter is the use of compiled expression information, e.g., a sequence database, which assembles information about expression levels of known sequences in cells whose sensitivity status is known. In certain embodiments of the invention, the reference cell population is enriched for CD11b+Gr1+ myeloid cells. In certain embodiments of the invention, the reference cell population is enriched for tumor cells.

Tumors resistant to treatment with VEGF antagonists can also be identified using the diagnostic marker sets provided in copending application Ser. No. 11/692,682 filed on Mar. 28, 2007. For example, a marker set can include two or more, three or more, four or more, five or more, six or more, seven or more, eight or more, nine or more, ten or more, twelve or more, thirteen or more, fourteen or more, fifteen or more, twenty or more, or the entire set, of molecules. The molecule is a nucleic acid encoding a protein or a protein with an altered expression and/or activity, and is selected from the following: Notch2, DMD8, MCP-1, ITGB7, G-CSF, IL-8R, MIP2, MSCA, GM-CSF, IL-1R, Meg-SF, HSP1A, IL-1R, G-CSFR, IL10-R1, Erb-2.1, Caveolin3, Semcap3, INTG4, THBSP-4, ErbB3, JAM, Eng, JAM, Eng, JAM-2, Pecam1, Tlr3, neutropil elastase, CD14, expi, Il-13R, LDLR, TLR-1, RLF, Endo-Lip, SOCS13, FGF13, IL-4R, THBS1, Crea7, Aquaporin-1, SCF38, APOE, FABP, IL-11R, IL-1RII, IFN TM1, TNFRSF18, WNT5A, Secretory carrier membrane 1, HSP86, EGFR, EphRB2, GPCR25, HGF, Angiopoietin Like-6, Eph-RA7, Semaphorin Vlb, Neurotrophin 5, Claudin-18, MDC15, ECM, ADAMTS7B, NCAM-140, Fibronectin type III, WIP, CD74, ICAM-2, Jagged1, ltga4, ITGB7, TGF-BII-R, TGFb IEP, Smad4, BMPR1A, CD83, Dectin-1, CD48, E-selectin, IL-15, Suppressor of cytokine signaling 4, Cytor4, CX3CR1, IGF2, HSP9A, FGF18, ELM1, Ledgfa, scavenger receptor type A, Macrophage C-type lectin, Pigr3, Macrophage SRT-1, G protein-coupled receptor, ScyA7, IL-1R2, IL-1 inducible protein, IL-1beta, ILIX Precuror, TGF-B, FIZZ1, Wfs1, TP 14A, EMAP, SULF-2, Extracellular matrix 2, CTFG, TFPI, XCP2, Ramp2, ROR-alpha, Ephrin B1, SPARC-like 1 and Semaphorin A. In one embodiment of the invention, an antibody is provided that detects the protein. In one embodiment, the molecules are derived from CD11b+Gr1+ cells and include, e.g., IL-13R, TLR-1, Endo-Lip, FGF13, IL-4R, THBS1 and Crea7. In another embodiment, the molecules are derived from resistant tumors and include, e.g., MSCA, MIP2, IL-8R, G-CSF, IL10-R2, THBSP-4, and JAM-2.

### C. PHARMACEUTICAL COMPOSITIONS AND ADMINISTRATION

The IL-17 antagonists, such as anti-IL-17 antibodies, of the present invention, alone or in combination with other therapeutic agents, are administered to a human patient, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes, and/or subcutaneous administration.

In certain embodiments, the treatment of the invention involves the combined administration of a IL-17 antagonist and a VEGF antagonist and/or one or more myeloid cell reduction agent or chemotherapeutic agent. In one embodiment, additional anti-cancer agents are present, e.g., one or more different anti-angiogenesis agents, one or more chemotherapeutic agents, etc. The invention also contemplates administration of multiple inhibitors, e.g., multiple antibodies to the same antigen or multiple antibodies to different proteins of the invention. In one embodiment, a cocktail of different chemotherapeutic agents is administered with the IL-17 antagonist herein. The combined administration includes coadministration, using separate formulations or a single pharmaceutical formulation, and/or consecutive administration in either order. For example, a VEGF or a G-CSF antagonist may precede, follow, alternate with administration of the IL-17 antagonist, or may be given simultaneously therewith. In one embodiment, there is a time period while both (or all) active agents simultaneously exert their biological activities.

For the prevention or treatment of disease, the appropriate dosage of the agent of the invention will depend on the type of disease to be treated, as defined above, the severity and course of the disease, whether the inhibitor is administered for preventive or therapeutic purposes, previous therapy, the patient's clinical history and response to the inhibitor, and the discretion of the attending physician. The inhibitor is suitably administered to the patient at one time or over a series of treatments. In a combination therapy regimen, the compositions of the invention are administered in a therapeutically effective amount or a therapeutically synergistic amount. As used herein, a therapeutically effective amount is such that administration of a composition of the invention and/or co-administration of IL-17 antagonist and one or more other therapeutic agents, results in reduction or inhibition of the targeting disease or condition. The effect of the administration of a combination of agents can be additive. In one embodiment, the result of the administration is a synergistic effect. A therapeutically synergistic amount is that amount of IL-17 antagonist and one or more other therapeutic agents, e.g., a IL-17 antagonist and optionally a myeloid cell reduction agent, a chemotherapeutic agent and/or an anti-cancer agent, necessary to synergistically or significantly reduce or eliminate conditions or symptoms associated with a particular disease.

Depending on the type and severity of the disease, about 1 μg/kg to 50 mg/kg (e.g. 0.1-20 mg/kg) of an IL-17 antagonist, VEGF antagonist, G-CSF antagonist, myeloid cell reduction agent, a chemotherapeutic agent, or an anti-cancer agent is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. Typically, the clinician will administered a molecule(s) of the invention until a dosage(s) is reached that provides the required biological effect. The progress of the therapy of the invention is easily monitored by conventional techniques and assays.

For example, preparation and dosing schedules for angiogenesis inhibitors, e.g., anti-VEGF antibodies, such as AVASTIN® (Genentech), may be used according to manufacturers' instructions or determined empirically by the skilled practitioner. In another example, preparation and dosing schedules for such chemotherapeutic agents may be used according to manufacturers' instructions or as determined empirically by the skilled practitioner. Preparation and dosing schedules for chemotherapy are also described in Chemotherapy Service Ed., M. C. Perry, Williams & Wilkins, Baltimore, Md. (1992).

The efficacy of the treatment of the invention can be measured by various endpoints commonly used in evaluating neoplastic or non-neoplastic disorders. For example, cancer treatments can be evaluated by, e.g., but not limited to, tumor regression, tumor weight or size shrinkage, time to progression, duration of survival, progression free survival, overall response rate, duration of response, quality of life, protein expression and/or activity. Because the anti-angiogenic agents described herein target the tumor vasculature and not necessarily the neoplastic cells themselves, they represent a unique class of anticancer drugs, and therefore can require unique measures and definitions of clinical responses to drugs. For example, tumor shrinkage of greater than 50% in a 2-dimensional analysis is the standard cut-off for declaring a response. However, the inhibitors of the invention may cause inhibition of metastatic spread without shrinkage of the primary tumor, or may simply exert a tumouristatic effect. Accordingly, approaches to determining efficacy of the therapy can be employed, including for example, measurement of plasma or urinary markers of angiogenesis and measurement of response through radiological imaging.

In another embodiment of the invention, an article of manufacture containing materials useful for the treatment of the disorders or diagnosing the disorders described above is provided. The article of manufacture comprises a container, a label and a package insert. Suitable containers include, for example, bottles, vials, syringes, etc. The containers may be formed from a variety of materials such as glass or plastic. In one embodiment, the container holds a composition which is effective for treating the condition and may have a sterile access port (for example the container may be an intravenous solution bag or a vial having a stopper pierceable by a hypodermic injection needle). At least one active agent in the composition is VEGF modulator and at least a second active agent is a myeloid cell reduction agent and/or a chemotherapeutic agent. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution.

Further details of the invention are illustrated by the following non-limiting examples. In the examples below, the student's t-test was used to determine significant differences in all experiments. P values of <0.05 were considered significant.

### Example 1

### Secreted Protein Profile of Anti-VEGF Resistant and Anti-VEGF Sensitive Tumor Cells

In order to identify tumor derived factors responsible for establishing and instructing its microenvironment, the profile of secreted proteins between previously established anti-VEGF resistant and sensitive tumor cell lines were compared. Mouse tumor cell lines (EL4, Tib-6) were obtained from the American Type Culture Collection (ATCC). EL4 is a T-cell lymphoma cell line that is anti-VEGF-resistant, while Tib6 is a B-cell lymphoma cell line that is anti-VEGF-sensitive. These were both cultured in DMEM (Invitrogen, Carlsbad, Calif.) supplemented with L-glutamine, 10% fetal bovine serum (FBS) (Sigma, St. Louis, Mo.) and maintained at 37° C. in a 5% CO₂, 80% humidity incubator.

EL4 and Tib-6 cell lines conditioned media were collected after the tumor cell lines were grown in 6-well plates at a density of 1×10⁶/ml in reduced serum DMEM (1% FBS) for 72 hrs. Cell viability and total cell number were measured using Vi-Cell XR (Beckman Coulter, Fullerton, Calif.) to account for changes in cell number during the conditioning period. All data were normalized by cell number at the end of the conditioning period.

Tumor cell secreted factors contained within the condition media were interrogated with a panel of antibodies specific for 32 mouse cytokines and growth factors (the BioRad cytokine bead assay described in Example 2). In an attempt to model in vivo conditions in which tumor cells come into contact with infiltrated stromal cells, we used the co-culturing assay in order to study the interaction between tumor and the major stromal cell type, fibroblasts. This is the simplest system to study cell-cell interaction. Normal skin fibroblasts isolated from mice were used in the co-culture studies to most closely mimic the in vivo setting. In the co-culture assay, detect changes in gene expression of either cell type are easily detected which appear when different cell types are in contact or within close proximity

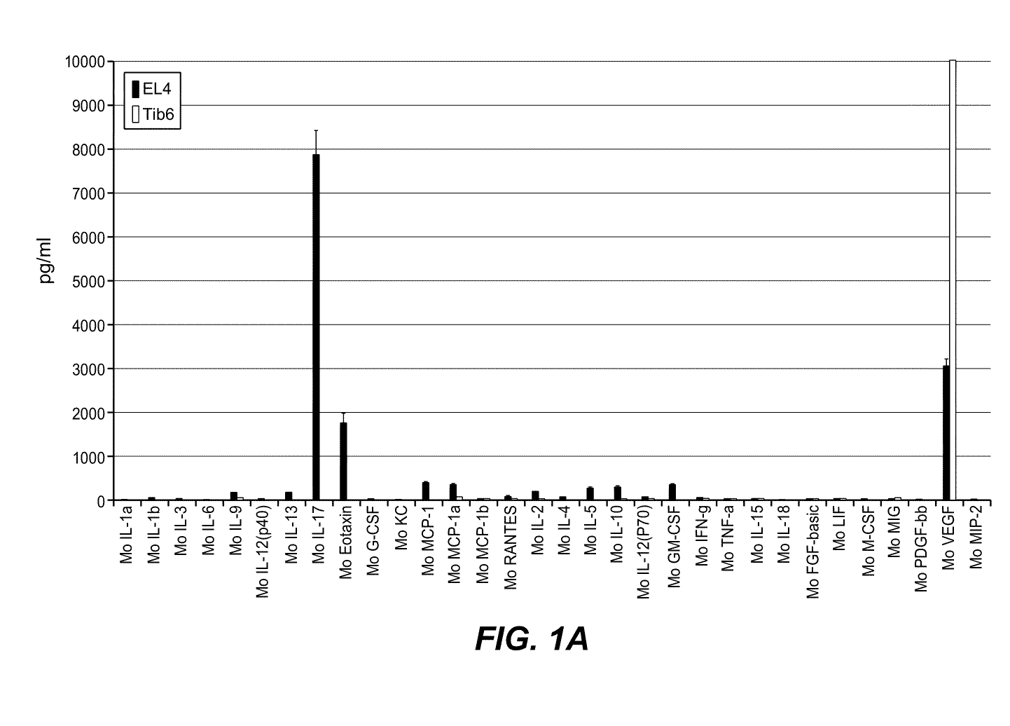

As shown in FIG. 1A, the results demonstrate that Il-17 is the most abundant secreted factor found in anti-VEGF resistant (EL4) vs. sensitive (Tibio) tumor cells in vitro. FIG. 1B shows that IL-6 and G-CSF levels are elevated in co-cultures of anti-VEGF refractory EL4 cells and normal skin fibroblasts (NSF). These findings indicate that the anti-VEGF resistant and sensitive cell lines differ in their secreted protein profile, most notably IL-17 as the most abundantly expressed cytokine in the resistant EL4 cell line. Additionally, the pro-inflammatory cytokines, IL-6 and G-CSF, are strongly up-regulated upon co-culturing the resistant tumor cell line with NSF, suggesting that cell-cell interaction between tumor and stromal-fibroblast cells can induce the expression of pro-inflammatory cytokines.

### Example 2

### Tumor Cells Induce Expression of Proinflammatory Genes in Fibroblasts Via a Paracrine Mechanism

RNA Sample Preparation and Quantitative Reverse Transcriptase-PCR (qRT-PCR) Analysis:

Total DNA-free RNA was isolated with the RNeasy kit (Qiagen, Germany) according to the manufacturer's protocol. One-step quantitative reverse transcription-PCR was done in a total volume of 50 μL with SuperScript III Platinum One-Step qRT-PCR Kit (Invitrogen, Carlsbad, Calif.) or with TaqMan One-Step RT-PCR Master Mix (Applied Biosystems, Foster City, Calif.). The following TaqMan Gene Expression Assay primers and probe mixes were used for the following murine genes: IL-17 (assay ID: Mm00439619_m1), IL-6 (assay ID: Mm01210733_m1), Bv8 (assay ID: Mm00450080_m1), G-CSF (assay ID: Mm00438334_m1), MMP-9 (assay ID: Mm00442991_m1), S100a8 (assay ID: Mm00496696_g1), S100a9 (assay ID: Mm00656925_m1), GAPDH (assay ID: Mm99999915_g1). Analyses were carried out on a standard ABI 7500 machine (Invitrogen) according to the manufacturer's recommended protocols.

### Cytokine Bead Assays and ELISA:

EL4 cells (1×106 cells/ml) and normal skin fibroblasts (NSF) (3.3×10⁵ cells) were cultured either alone or together at 1:3 (NSF/EL4) ratio in 6-well plates for 72 hrs in triplicate; supernatants were collected and analyzed with Bio-Plex Pro™ Magnetic Cytokine, Chemokine, and Growth Factor Assays system (BioRad, Hercules, Calif.). Cells were cultured in reduced serum medium (1% FBS). Murine IL-17A, G-CSF, IL-6 levels were measured by Quantikine ELISA kit (R&D Systems, Minneapolis, Minn.).

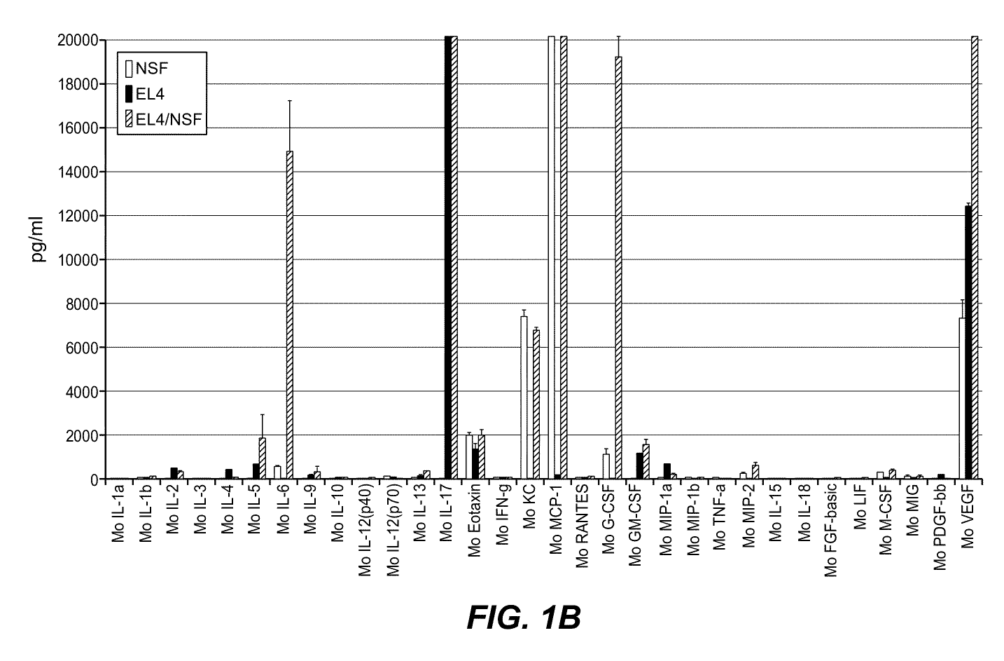

GFP-labeled EL4 tumor cells were co-cultured with normal skin fibroblasts (NSF) for 72 h, followed by FACS isolation of tumor cells from fibroblasts and analysis of gene expression by qRT-PCR. As shown in FIG. 2A, G-CSF (Csf3), and in FIG. 2B, IL-6 expression are induced and were observed in normal fibroblasts when in co-cultured with the anti-VEGF resistant cell line EL4 vs. mono-cultured cells. Pre-sort analysis was performed to control for potential artifacts introduced by FACS sorting. In order to determine the cellular source of G-CSF and IL-6 expression, GFP labeled tumor cells were FACS sorted following co-culture with unlabelled fibroblasts and profiled changes in gene expression in these cell types by qRT-PCR. This data revealed that the cell-cell interaction resulted in the induction of both IL-6 and G-CSF in the fibroblast compartment versus tumor cells upon co-culturing. Similarly, when NSFs were stimulated with EL4-conditioned media, an induction of G-CSF and IL-6 expression in fibroblasts was also observed (data not shown), suggesting that cell-cell interaction is not strictly required and that tumor cell secreted factors are sufficient to elicit an up-regulation of G-CSF and IL-6 in NSFs. Together this data suggest that tumor cells may be instructing neighboring fibroblasts into expression and secretion of pro-inflammatory cytokines including G-CSF and IL-6 via a paracrine mechanism.

### Example 3

### IL-17 Neutralization Inhibits EL4 Induced G-CSF Expression in Fibroblasts

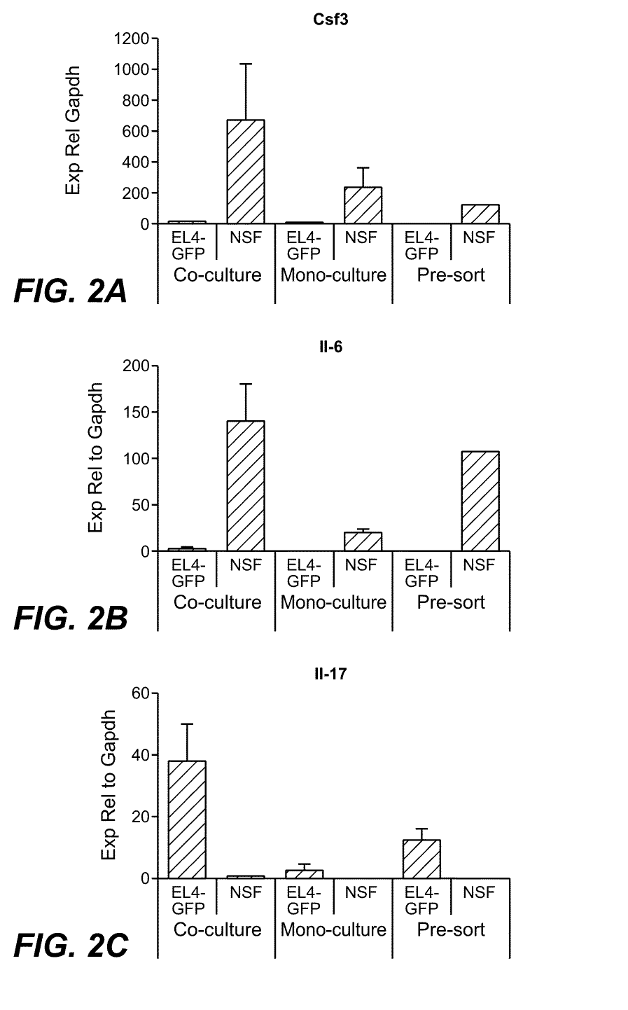

In order to address whether the observed G-CSF induction in NSF may be IL-17 dependent, EL4 conditioned medium was collected as described in Example 1, then pre-incubated with neutralizing antibodies against IL-17, allowing for neutralization of the targeted soluble factors before adding to NSFs plated in 96-well clusters. Neutralizing antibodies against IL-17 and two other known inducers of G-CSF (TNF-α and IL-1β) of varying concentrations was incubated with EL4/fibroblasts in co-culture for 24 h at 37° C. in a total volume of 200 μl. After this incubation, 50 μL of supernatant was collected from each well, diluted with 50 μL of ELISA diluent and tested for mG-CSF levels by ELISA. As shown in FIG. 3, neutralizing antibodies against IL-17 provided the most significant reduction in the levels of secreted G-CSF in conditioned media of EL4/fibroblasts co-cultures, suggesting that IL-17 may be a dominant tumor-derived factor responsible for inducing pro-inflammatory cytokine secretion in associated normal fibroblasts.

### Example 4

### IL-17 Function in the Tumor Microenvironment

Female mice (6- to 12-weeks-old) were used as indicated: C57B16.IL-17RC−/−, C57B16. WT littermates were bred and maintained at Genentech, Inc. under specific pathogen-free conditions. Female WT C57B16 mice were purchased from Charles River Laboratory (Hollister, Calif.). Procedures involving animals were reviewed and approved by the Institutional Animal Care and Use Committee, Genentech, Inc., and conform to the relevant regulatory standards. EL4 tumor cells were cultured as described in Example 1.

The tumor mouse models used in this experiment are as described as follows: EL4 tumor cells (2.0×10⁶) in 100 ul of growth factor-reduced matrigel (BD BioScience) were subcutaneously inoculated in the dorsal flank of mice of different genotypes, either wildtype (C57/BL6 WT) or IL-17 receptor knock-out (IL-17rc KO). Antibodies were IP injected twice per week at the doses indicated in the corresponding figure legends. Treatments with the control antibody, anti-Ragweed, anti-VEGF mAb B20-4.1.1 (Liang et al., 2006) or anti-IL-17A were initiated 2 days after tumor cell inoculation. All tumor growth experiments were performed at least three times and conducted in accordance with the Guide for the Care and Use of Laboratory Animals. Tumor volumes were calculated every other day using the ellipsoid volume formulas (0.5×L×W2, where L is length and W is width).

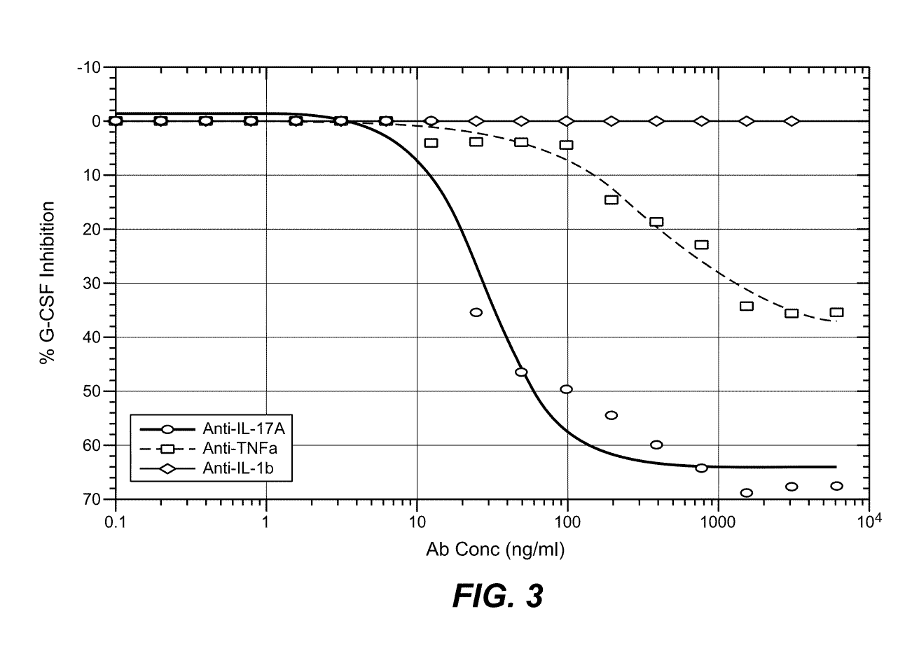

FIG. 4A shows growth of EL4 tumors in C57/BL6 WT and Il-17rc−/− mice treated with control antibody (anti-Ragweed, 10 mg/kg, intraperitoneally (IP), twice weekly) or anti-VEGF (10 mg/kg, IP, twice weekly). Treatment was initiated 48 h after tumor cell inoculation. Data are shown as mean±SEM. In the control treatment group, the terminal EL4 tumor volume was reduced by ˜50% in IL-17rc−/− mice as compared to WT suggesting that IL-17 signaling to the host stroma plays a significant role in tumor growth. And whereas mono-therapy with anti-VEGF has a slight effect on EL4 tumor shrinkage in WT mice, it resulted in almost ˜80% tumor growth inhibition in IL-17rc−/− mice. Data are shown as mean±SEM. *indicates significant difference (P<0.0001) between EL4 tumors in WT and Il-17rc−/− animals treated with anti-VEGF.

FIG. 4B shows growth of EL4 tumors in C57/BL6 WT mice treated with control antibody (anti-Ragweed), anti-VEGF, anti-mIL-17, and combination anti-mIL-17/anti-VEGF. All antibodies were administered at 10 mg/kg, intraperitoneally (IP), twice weekly. Treatment was initiated 48 h after tumor cell inoculation. Data are shown as mean±SEM. When anti-VEGF was administered alone, terminal tumor volume was marginally reduced, whereas when anti-IL-17 was administered in conjunction with anti-VEGF, tumor volume was reduced by ˜50%, further suggesting that inhibition of IL-17 renders susceptibility to anti-VEGF treatment. Data are shown as mean±SEM (Shojaei et al 2009). (*) indicates significant difference (P<0.05) between EL4 tumors treated with anti-VEGF and in combination with anti-mIL-17. Together, FIGS. 4A and 4B demonstrate that IL-17 function in the tumor microenvironment may promote tumor growth and may be required for resistance to anti-VEGF treatment.

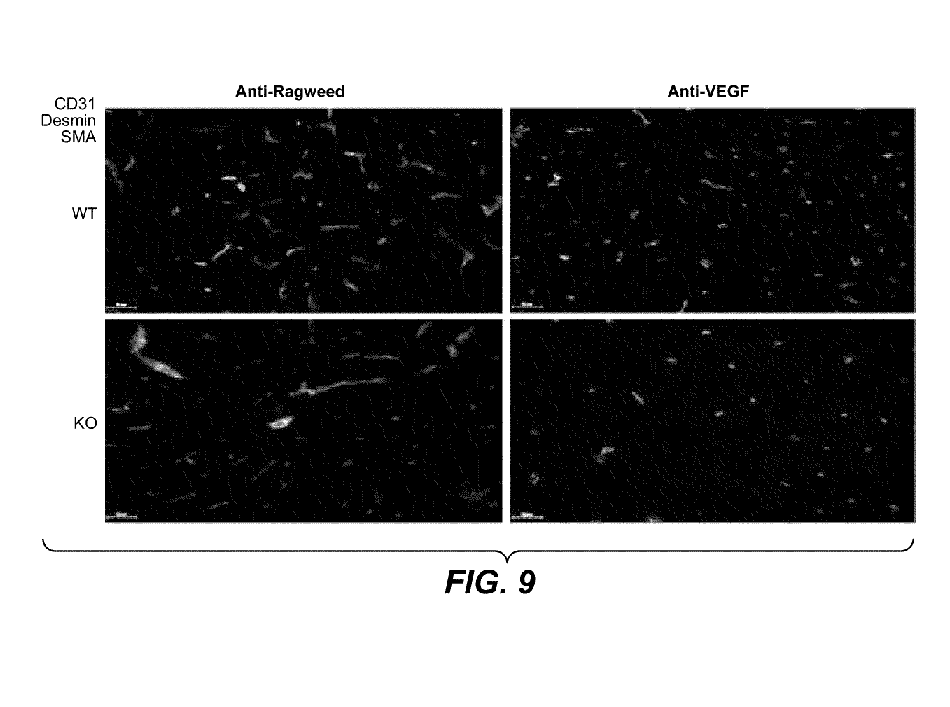

Furthermore, the role of IL-17 in mediating tumor resistance to VEGF inhibition was confirmed when a treatment-sensitive tumor cell line, Tib-6, was transduced with mouse IL-17A (denoted Tib6-IL17) and tested for its response to anti-VEGF treatment in immunodeficient (nu/nu) recipient mice. There were significantly higher levels of both circulating and tumor IL-17A compared to control, neomycin-transduced Tib6 (denoted as Tib6-neo) tumor bearing mice. Correspondingly, higher levels of G-CSF were also detected in Tib6-IL17 tumors and these tumors recruited more CD11b+Gr1+ cells in vivo than did Tib6-neo tumors. Although, implanted Tib6-IL-17 tumors did not exhibit significant increase in tumor growth rate compared to Tib6-neo tumor, these tumors were significantly more resistant to anti-VEGF treatment across 4-independent stable Tib-6/IL-17 clones compared to the 2-Tib6/neo control clones tested (FIG. 11). Furthermore, this gain-of-function data in nu/nu mice indicates that IL-17 effector function can occur independently of additional inputs from T-cells suggesting that IL-17 function alone is necessary and sufficient in mediating a pro-inflammatory network driving resistance to anti-VEGF treatment.

### Example 5

### IL-17 Signaling Experiments in EL4 Tumor Bearing Mice

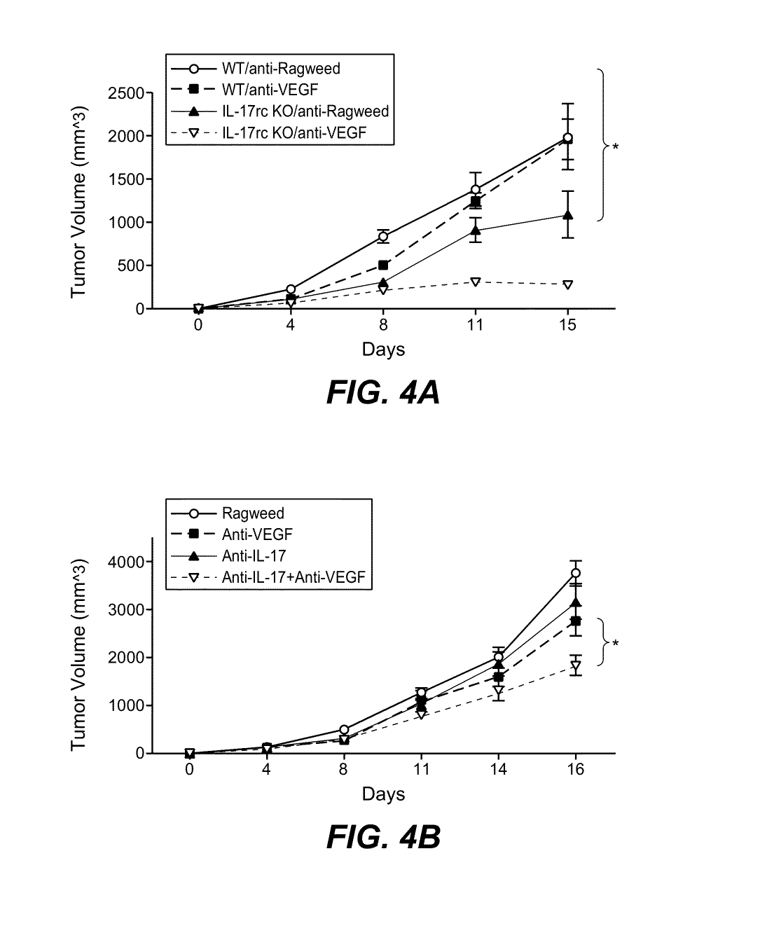

Circulating cytokine levels in blood serum of naïve and tumor bearing mice were determined as described in Example 2. Bv8 concentrations were measured by ELISA as described previously (Shojaei et al 2009). The tumor mice models used in this experiment are as described in Example 4. FIG. 5A-C shows serum levels of mG-CSF, mBv8 and mIL-17A in EL4-bearing WT and Il-17rc−/− mice treated with either control anti-Ragweed antibody (Rag, 10 mg/kg) or anti-VEGF antibody (B20, 10 mg/kg). Data are shown as means±SD. This data indicates that the levels of IL-17 and G-CSF are associated with the presence of tumor when comparing cytokine levels in tumor-bearing versus naïve mice. Secondly, the levels of both G-CSF and the pro-angiogenic factor Bv8 appears to be dependent upon IL-17 signaling to host cells as both factors return to naïve levels in IL-17RC−/− hosts. Furthermore, there is no decrease in the level of IL-17 found in circulation comparing WT to IL-17RC−/− mice suggesting that IL-17 expression is tumor intrinsic. Together, this experiment data demonstrates that IL-17 signaling to the host stromal cells regulates levels of pro-inflammatory/pro-angiogenic cytokines in tumor bearing mice.

Using EL4 tumor bearing mice as described above, WBCs were isolated from tumor bearing mice and CD11b+Gr1+ cells were isolated from the mouse spleen as follows: single cell suspensions were prepared from spleens isolated from naïve and tumor bearing mice and the CD11b+Gr1+ population was sorted by first labeling cells with anti-Gr-1-PE conjugate followed by anti-PE microbeads (Miltenyi Biotech) according to protocols provided by the manufacturer. For quality control, an aliquot of the sorted cells was stained with anti-CD11b and anti-Gr1 and analyzed by FACS analysis to ensure the purity (more than 90%) of CD11b+Gr1+ cells.

Flow cytometry of bone marrow mononuclear cells (BMNCs), peripheral blood mononuclear cells (PBMNCs), and tumor cells were harvested from mice implanted with tumors. Tumors from control and anti-VEGF-treated mice were isolated and single cell suspensions were obtained by mincing tumors with razor blades and homogenizing by mechanical disruption and digestion with Collagenase/Dispase and Dnase (Roche, Basel, Switzerland) at 1 mg/ml in growth media for 1 hr at 37° C. Red blood cells were lysed using ACK (Lonza, Basel, Switzerland) lysis buffer, followed by staining with rat anti-mouse CD11b and Gr-1 antibodies (BD Bioscience, San Jose, Calif.). To exclude dead cells, propidium iodide (Sigma, St. Louis, Mo.) was added to all samples before data acquisition on the LSRIIB FACS instrument (BD Biosciences) and analysis using FlowJo software (Tree Star, Ashland, Oreg.).

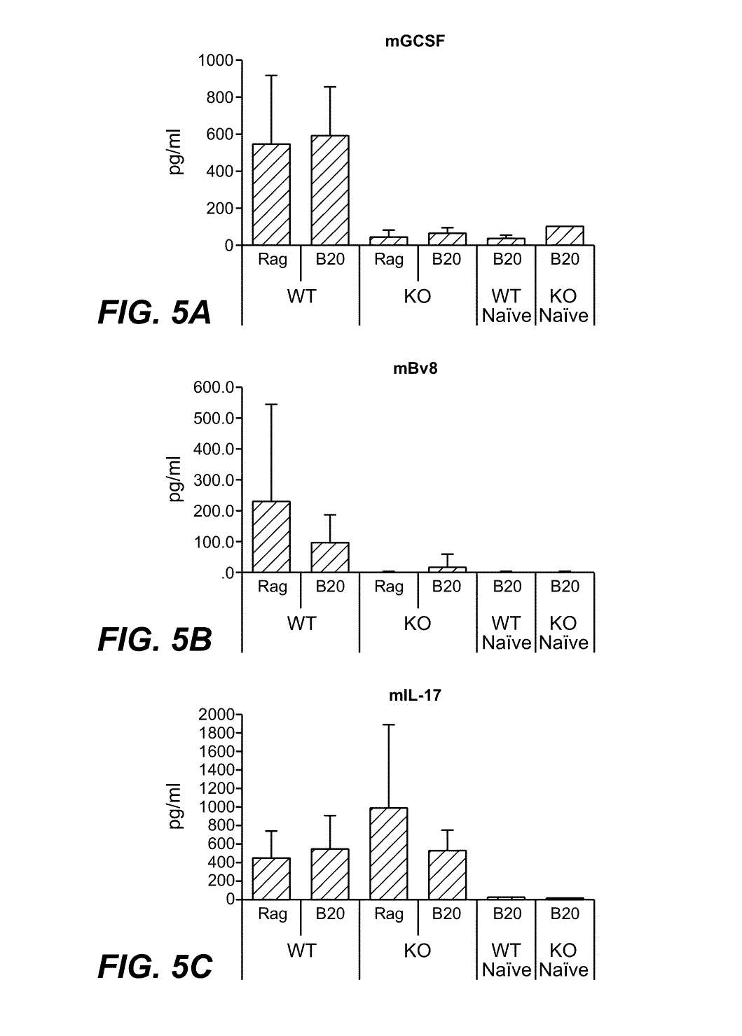

Immune-suppressive immature myeloid cells are defined as CD11b+/Gr1+ double-positive cells and are associated with tumor expansion, reviewed in (Gabrilovich & Nagaraj 2009). Since G-CSF and Bv8 have been reported to recruit and mobilize CD11b+Gr1+ cells and to confer tumor resistance to anti-VEGF antibodies (Shojaei et al 2009), it was investigated whether the elevated G-CSF and Bv8 levels in EL4 tumor bearing mice are likewise responsible for recruitment of CD11b+Gr1+ cells in mediating resistance to anti-VEGF Immune-suppressive immature myeloid cells are defined as Gr1+/CD11b+ double-positive cells (FIG. 6) and quantified, as shown in FIG. 7A-C, and demonstrates the mobilization of CD11b+Gr1+ cells into the circulation as determined by flow cytometric analysis in tumor bearing mice compared to naïve mice. Furthermore, CD11b+Gr1+ mobilization is dependent on IL-17 signaling to the tumor microenvironment as indicated by the significant reduction in CD11b+Gr1+ cells found in circulation of tumor-bearing IL-17RC−/− mice. Since previous studies suggest that splenic CD11b+Gr1+ cells contribute to tumor expansion (Kusmartsev & Gabrilovich 2002), (Bronte et al 2000), the spleens of tumor bearing mice were examined and a reduction of splenic CD11b+Gr1+ cell in IL-17RC−/− compared to WT hosts was observed. Quantification of flow cytometry results in FIG. 7A-C also demonstrates that less CD11b+Gr1+ cells are recruited to tumors in IL-17RC−/− compared to WT hosts. Together, this experiment data demonstrates that IL-17 signaling to the host stroma may be required for the mobilization and tumor infiltration of CD11b+Gr1+ immune-suppressive immature myeloid cells in EL4 tumor bearing mice.

### Example 6

### IL-17 May be Necessary for Tumor Promoting Function

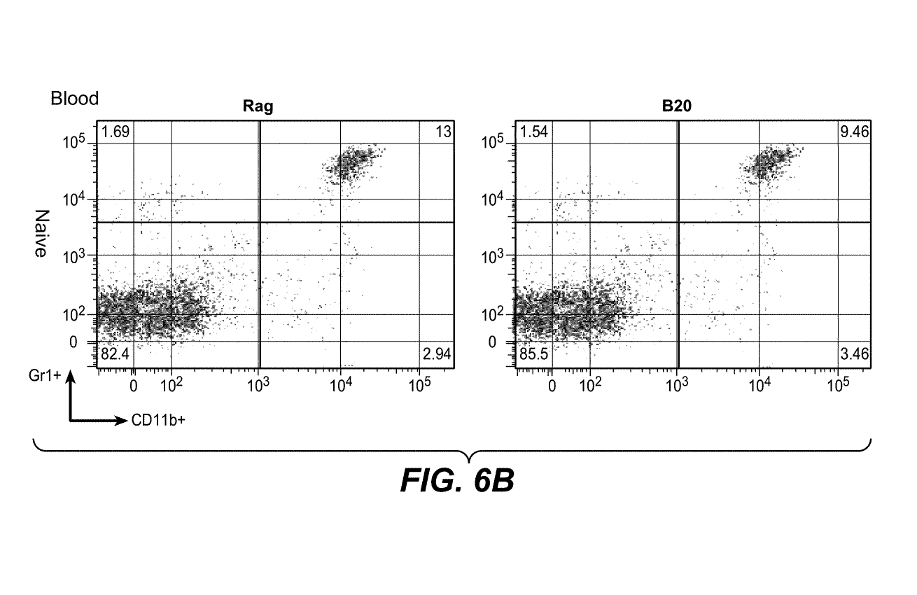

To further explore the tumor promoting phenotype ascribed to splenic CD11b+Gr1+ cells from resistant tumor bearing mice and to gain further understanding of whether IL-17 signaling plays a role in priming the phenotype of host CD11b+Gr1+ cells, Gr1+ cells were isolated from spleens of EL4 tumor-bearing mice were performed as described in Example 5. Following verification of splenic CD11b+Gr1+ cell purity, these cells were cultured overnight in the absence or presence of LPS followed by qRT-PCR analysis for the expression of pro-angiogenic genes such as Bv8 (FIG. 8A) and tumor promoting genes such as S100A8 (FIG. 8B), S100A9 (FIG. 8D) and MMP9 (FIG. 8C) by qRT-PCR. The CD11b+Gr1+ cells in WT tumor-bearing mice expressed higher levels of this subset of tumor-promoting genes compared to the CD11b+Gr1+ those found in the spleen of IL-17RC−/− hosts suggesting that IL-17 may be acting as a priming signal responsible for determining the tumor promoting phenotype of host CD11b+Gr1+ cells and that IL-17 signaling contributes to the anti-VEGF resistance phenotype of host CD11b+Gr1+ cells.

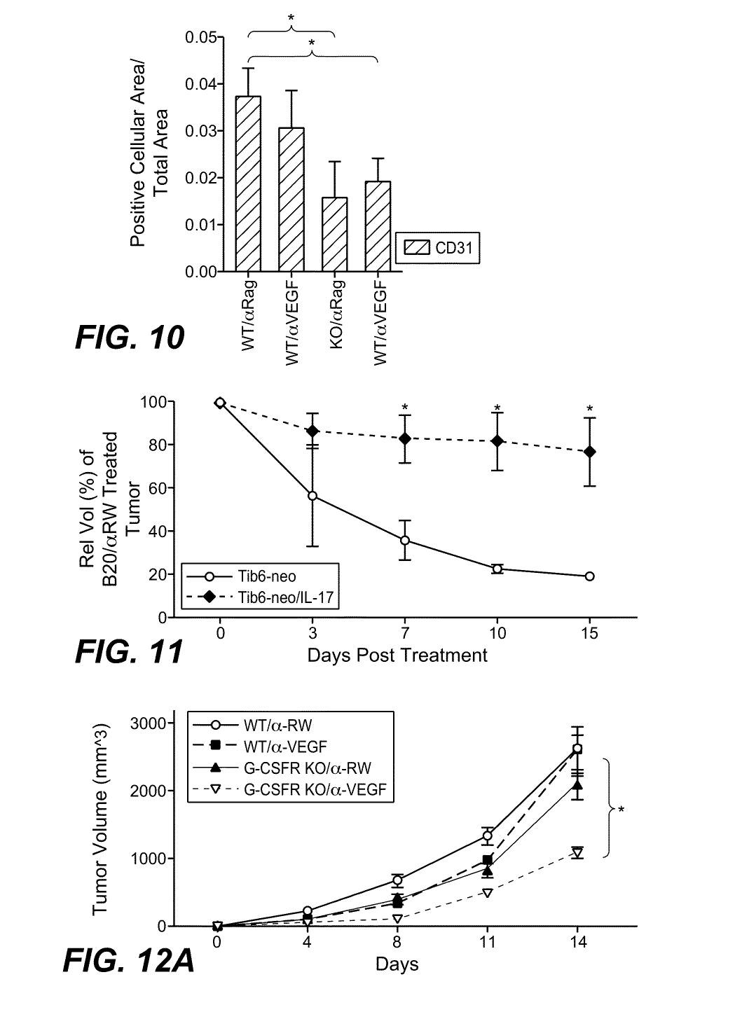

To test the requirement for G-CSF in mediating anti-VEGF refractoriness, the anti-VEGF resistant cell line, EL4, was implanted into syngeneic G-CSF receptor knockout C57BL/6 recipients (Csf3r−/−, hereafter referred to as GCSFR KO) where all stromal host cells are deficient for G-CSF signaling. While G-CSF signaling did not appear to alter the growth of EL4 tumors, it was observed that this signaling axis was indeed necessary in mediating tumor refractoriness to anti-VEGF treatment as well as the mobilization of CD11b+Gr1+ cells from the bone marrow and recruitment to the tumor microenvironment (FIG. 12A-C). Together this data definitively demonstrates the requirement for an IL-17-G-CSF signaling cascade in mediating anti-VEGF resistance via the mobilization and recruitment of CD11b+Gr1+ into the tumor microenvironment.

### Example 7

### Measuring Mean Vascular Density

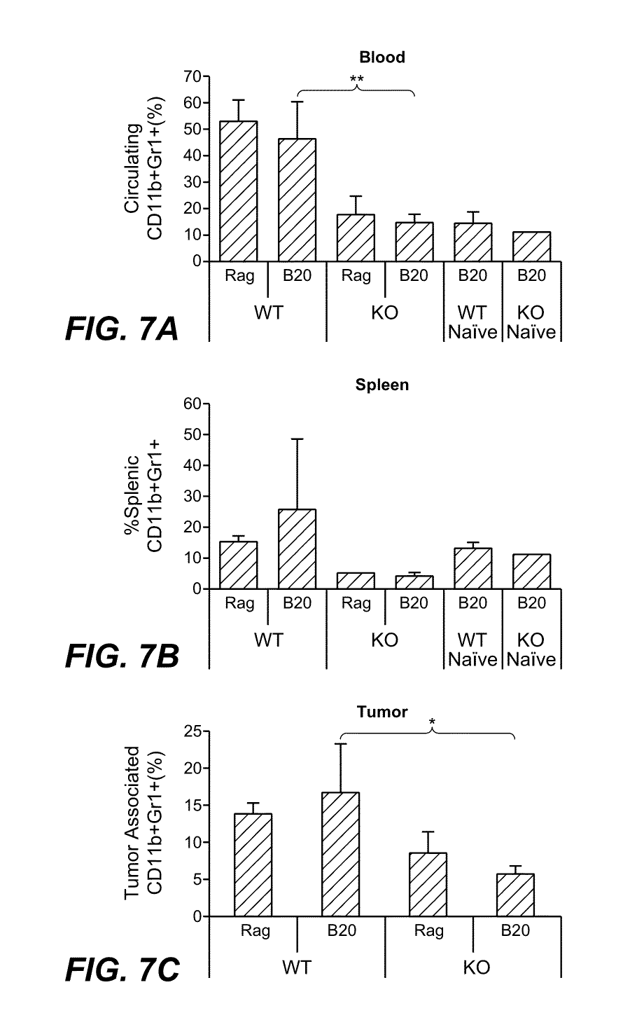

EL4 tumors derived from WT and IL-17rc KO animals, treated with control antibody (anti-Ragweed) or anti-VEGF antibody (B20) were immunostained as follows. Tumor samples were embedded in Optimum Cutting Temperature (OCT, Sakura Finetek) and frozen in dry-ice bath. Tumor sections were cut (10 um) in a cryostat (Leica Microsystem). Sections were dried at 20° C. for 1 h and then fixed in acetone for 10 min at −20° C. After air-drying, the nonspecific binding sites were blocked by incubation for 1 h at 20° C. in 10% normal donkey serum in 2% BSA/PBS (Jackson ImmunoResearch, West Grove, Pa.) followed by immuno-staining with antibodies diluted in 1.5% normal serum in 2% BSA/PBS. Tumor sections were stained with the following primary antibodies: rat anti-mouse CD31 antibody (Clone MEC13.3; BD Pharmingen) at 1:100 overnight at 4° C., anti-rabbit Desmin (Clone GTX15200, Genetex) at 1:400, and anti-mouse smooth muscle actin (SMA)-Cy3 conjugated (Sigma) at 1:400 overnight at 4° C. followed by secondary antibodies, anti-rat-Alexa-488 conjugate and anti-rabbit-Alexa 647 conjugate (Invitrogen) for 2 hrs at 20° C. The slides were counter stained with DAPI, washed and mounted in DAKO fluorescent mounting medium (DakoCytomation) Immunofluorescence images were collected on a Zeiss Axiolmager Z2 upright microscope (Zeiss) and TissueGnostics Slide scanner (TissueGnostics, Vienna, Austria). FIG. 9 shows the immunostained tissues.

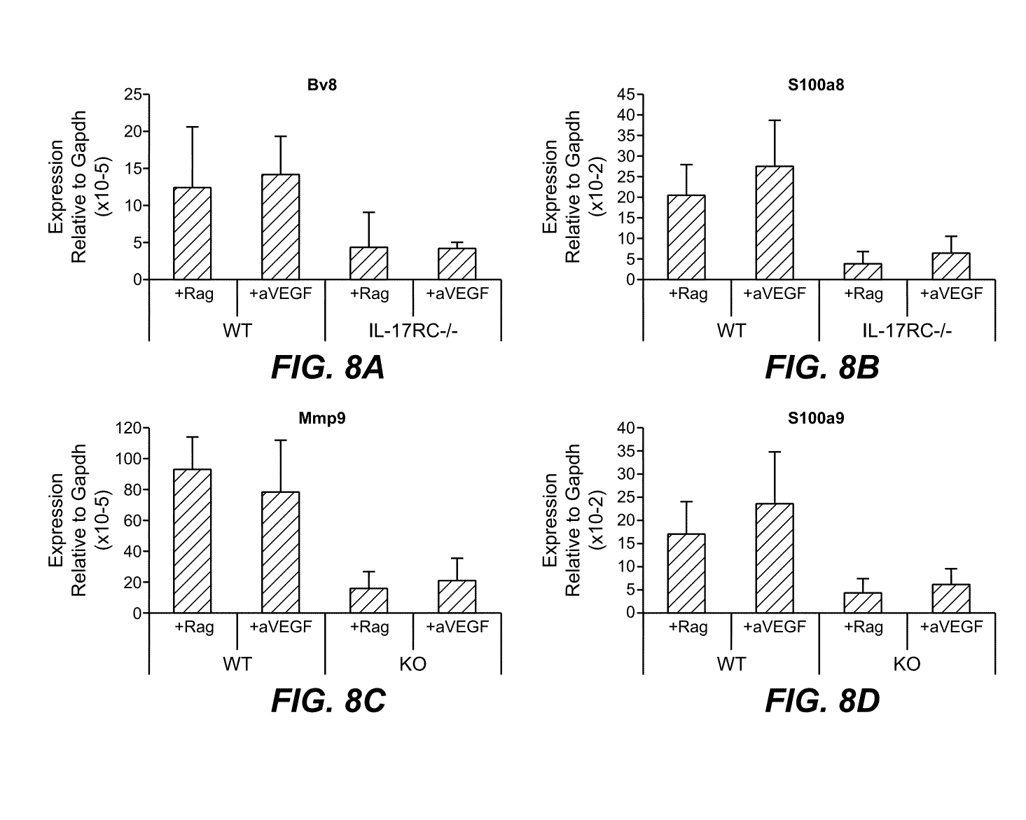

Quantification of the immunostaining is represented as average area of CD31-positive cells over total area of cells was performed as follows. Tumor mean vessel density (MVD) measurements were quantified from digital images captured on the TissueGnostics Slide scanner of CD31 stained sections using a 20× objective. The pixels corresponding to stained vessels were selected by using Definiens Tissue Studio Software (Definiens, N.J.). Whole tumor cross-sections and a total of 5 tumors per group were analyzed. The aggregate pixel vessel area, relative to the total picture area and total area analyzed, is reported as % positive cellular area/total surface area. FIG. 10 shows the quantification of tumor-angiogenesis measurement expressed as the MVD. The MVD in the resistant tumors is significantly decreased (p<0.05) in IL-17RC−/− compared to WT hosts suggesting that decreased MVD that is accompanied by suppression of tumor growth in IL-17rc KO mice is accompanied by decreased MVD for both the control and anti-VEGF treated groups. This data indicates that Il-17 signaling to the host microenvironment promotes new vessel growth.

### Example 8

### TH17-Cell Mediated Effects on Anti-VEGF Response

IL-17 is produced by the TH17 subset of CD4+ T cells and CD8+ T cells (Tc17). Since the infiltration of TH17 cells is associated with poor prognosis in human lung and colorectal cancers, the effect of tumor infiltrating TH17 cells in response to anti-VEGF treatment in syngeneic mouse lung and colon cancer models was tested.

Mouse tumor cell line CT-26 was obtained from the American Type Culture Collection and was cultured in RPMI (Invitrogen, Carlsbad, Calif.). Media was supplemented with L-glutamine, 10% fetal bovine serum (FBS) (Sigma, St. Louis, Mo.). CT-26 was cultured and maintained at 37° C. in a 5% CO2, 80% humidity incubator. CT-26 tumor cells (2.0×10⁶) in 100 μl of growth factor-reduced matrigel (BD BioScience) were subcutaneously inoculated in the dorsal flank of either C57B16.WT or C57B16.IL-17RC−/− (KO) mice. Antibodies were IP injected twice per week. Treatments with the control antibody, anti-Ragweed, anti-VEGF mAb B20-4.1.1 (Liang et al., 2006) or anti-IL-17A or anti-IL-17F were initiated 2 days after tumor cell inoculation. All tumor growth experiments were performed at least three times and conducted in accordance with the Guide for the Care and Use of Laboratory Animals. Tumor volumes were calculated every other day using the ellipsoid volume formulas (0.5×L×W2, where L is length and W is width).

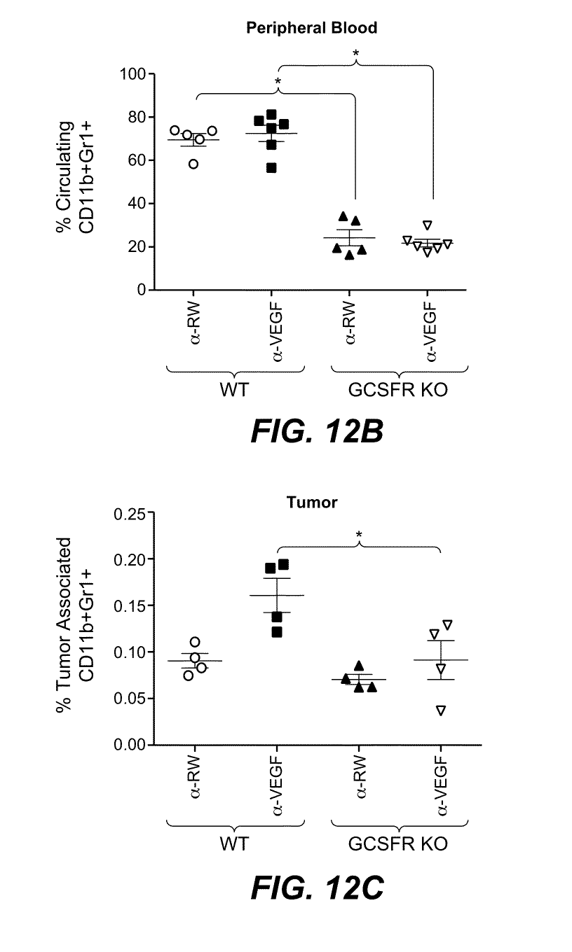

A significant reduction in tumor growth in the colorectal cancer cell line, CT-26, was observed when treated with anti-IL17 and anti-VEGF in combination versus anti-VEGF alone, as well as a significant decrease in tumor burden in Lewis Lung Carcinomas (LLC)-bearing Il-17rc−/− vs. WT littermates following anti-VEGF treatment (FIGS. 13A and B).

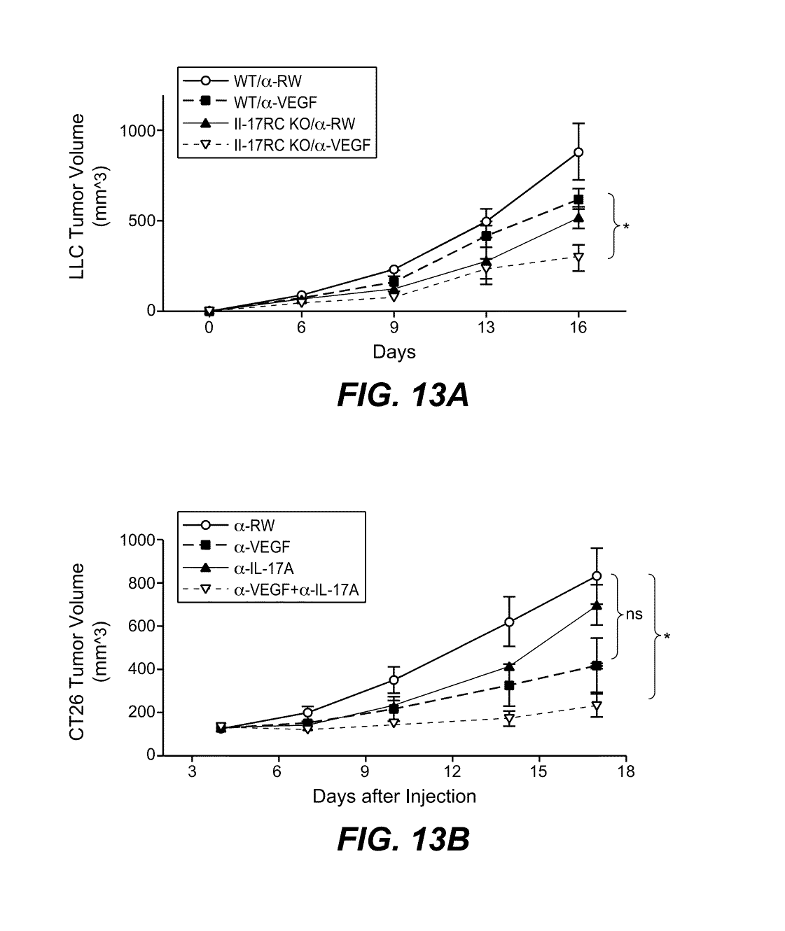

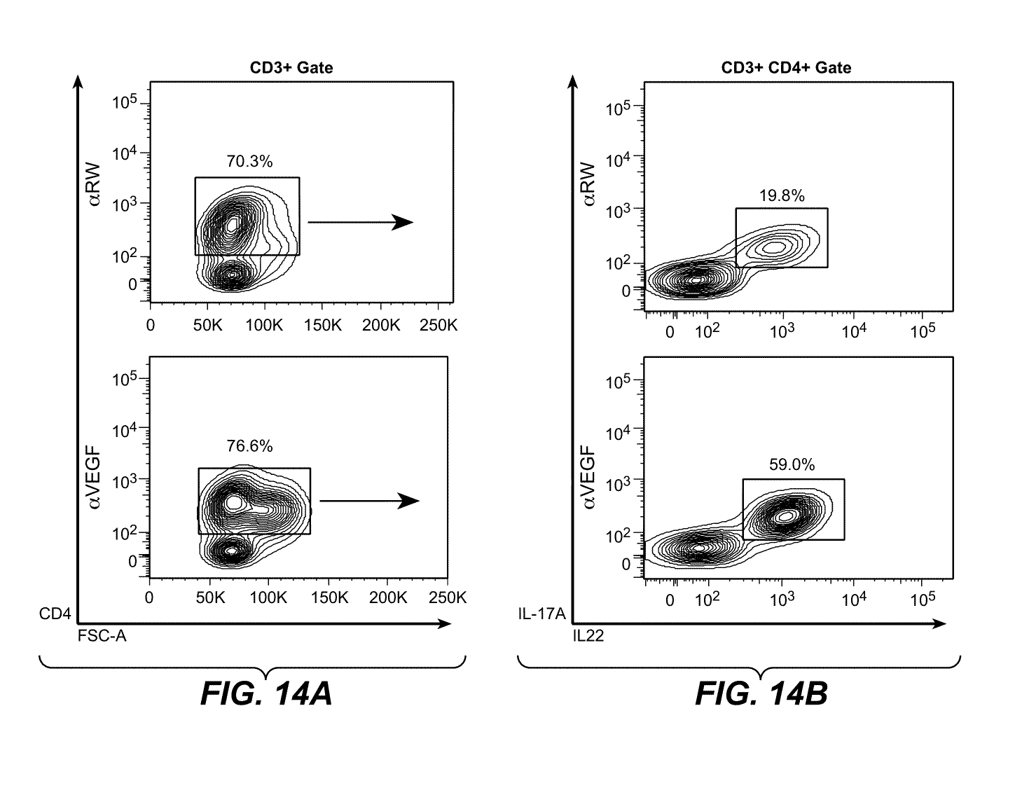

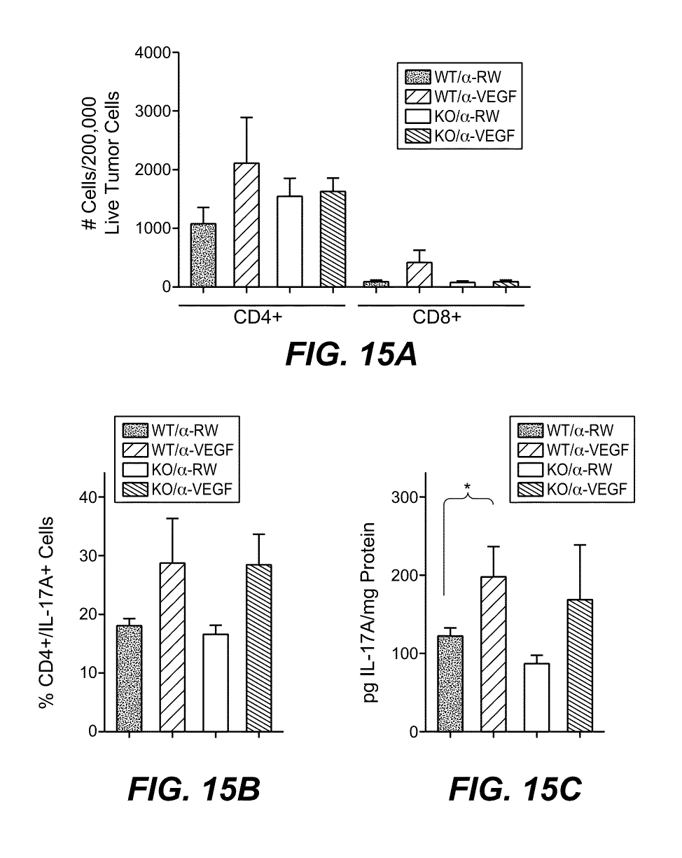

CT-26 tumor cells were harvested from mice implanted with tumors. Tumors from control- and anti-VEGF-treated mice were isolated and single cell suspensions were obtained by mincing tumors with razor blades and homogenizing by mechanical disruption and digestion with Collagenase/Dispase and Dnase (Roche, Basel, Switzerland) at 1 mg/ml in growth media for 1 hr at 37° C. To exclude dead cells, propidium iodide (Sigma, St. Louis, Mo.) was added to all samples before data acquisition on the LSRIIB FACS instrument (BD Biosciences) and analysis using FlowJo software (Tree Star, Ashland, Oreg.)(FIGS. 14A-B and FIG. 15B). Murine IL-17A and G-CSF were measured by Quantikine ELISA kit (R&D Systems, Minneapolis, Minn.). Murine Bv8 levels were measured using an ELISA assay developed at Genentech. (FIG. 15C and FIGS. 16A and C). The percentage of tumor infiltrating CD11b+Gr1+ cells were determined by flow cytometry. (FIG. 16B).

TIL analysis was performed as described previously. It was observed that the level of LLC tumor infiltrating CD3+ T-cells did not differ between WT and KO hosts (1.24%±0.18% and 1.28%±0.22% of live tumor cells respectively). However, consistent with recent reports indicating that anti-angiogenic therapies can increase tumor lymphocyte infiltration 30-32, it was observed that anti-VEGF treatment increased the number of both tumor infiltrating CD4+ and CD8+ though there were tenfold higher CD4+ than CD8+ T cells (FIGS. 15A-C). And while both these mature T-cell subpopulations are known to express IL-17, IL-17 expression was restricted CD4+ cells in LLC tumors (FIGS. 14A-B and FIG. 15A-C). All IL-17+CD4+ cells also expressed IL-22 (FIG. 14A-B), a hallmark cytokine for mature and terminally differentiated TH17 cells. An increase in IL17+IL-22+CD4+ population in LLC tumors was observed upon anti-VEGF treatment. The prevalence of these TILs was co-incident with an increase in both IL-17 and G-CSF levels in the tumor microenvironment and as well as an increase in recruitment of CD11b+Gr1+ myeloid cells and the intratumoral level of the pro-angiogenic factor, Bv8 (FIG. 16C). The downstream effects of TH17 tumor infiltration, however, were only observed in the presence of intact IL-17 signaling in the WT tumor-bearing hosts (FIG. 16A-C).

Recent studies have suggested that IL-17 plays a role in promoting tumor angiogenesis although most of these studies were performed with recombinant IL-17 protein or retroviral transduction of the IL-17 gene into tumors (Tartour, E. et al. Interleukin 17, a T-cell-derived cytokine, promotes tumorigenicity of human cervical tumors in nude mice. Cancer Res 59, 3698-704 (1999); Numasaki, M. et al. Interleukin-17 promotes angiogenesis and tumor growth. Blood 101, 2620-7 (2003)). The effect of endogenous tumor-infiltrating TH17 cells on tumor vasculature promotion in the syngeneic LLC tumor model was questioned. To this end, an enhanced depletion of tumor associated endothelial cells by anti-VEGF treatment in IL-17RC

KO hosts was observed as compared to WT, indicating that TH17 cells can similarly promote persistent angiogenesis in the face of VEGF blockade (FIG. 16A-C). Together, this data provides evidence for the requirement for tumor infiltrating TH17 cells and IL-17 signaling within the host microenvironment in mediating expression of the pro-inflammatory cytokine G-CSF and recruitment of pro-angiogenic CD11b+Gr1+ cells to further mediate resistance to anti-VEGF therapy.
Bronte V, Apolloni E, Cabrelle A, Ronca R, Serafini P, et al. 2000. Identification of a CD11b(+)/Gr-1(+)/CD31(+) myeloid progenitor capable of activating or suppressing CD8(+) T cells. Blood 96: 3838-46.
Gabrilovich D I, Nagaraj S. 2009. Myeloid-derived suppressor cells as regulators of the immune system. Nat Rev Immunol 9: 162-74.
Kusmartsev S, Gabrilovich D I. 2002. Immature myeloid cells and cancer-associated immune suppression. Cancer Immunol Immunother 51: 293-8.
Shojaei F, Wu X, Qu X, Kowanetz M, Yu L, et al. 2009. G-CSF-initiated myeloid cell mobilization and angiogenesis mediate tumor refractoriness to anti-VEGF therapy in mouse models. Proc Natl Acad Sci USA 106: 6742-7.

All references cited throughout the disclosure are hereby expressly incorporated by reference in their entirety. While the present invention has been described with reference to what are considered to be the specific embodiments, it is to be understood that the invention is not limited to such embodiments. To the contrary, the invention is intended to cover various modifications and equivalents included within the spirit and scope of the appended claims.

Throughout the present application, including the claims, the term “comprising” is used as an inclusive, open-ended transition phrase, which does not exclude additional, unrecited elements or method steps.

1. A method of inhibiting tumor angiogenesis, comprising administering to a human subject having a tumor previously treated with a vascular endothelial growth factor (VEGF) antagonist, an effective amount of an IL-17 antagonist, wherein the tumor is refractory to treatment with said VEGF antagonist.

2. A method of suppressing tumor growth, comprising administering to a human subject having a tumor previously treated with a VEGF antagonist, an effective amount of an IL-17 antagonist, wherein the tumor is refractory to treatment with said VEGF antagonist.

3. A method of tumor treatment, comprising administering to a human subject having a tumor previously treated with a VEGF antagonist, an effective amount of an IL-17 antagonist, wherein the tumor is refractory to treatment with said VEGF antagonist.

4. The method of claim 1, wherein the VEGF antagonist is an anti-VEGF antibody or fragment thereof.

5.-6. (canceled)

7. The method of claim 4, wherein the anti-VEGF antibody is bevacizumab comprising a variable heavy chain sequence of SEQ ID NO: 1 and a variable light chain sequence of SEQ ID NO: 2, or fragment or variant thereof.

8.-9. (canceled)

10. The method of claim 1, wherein the IL-17 antagonist is an anti-IL-17 antibody or fragment thereof or an anti-IL-17 receptor antibody or a fragment thereof.

11.-12. (canceled)

13. The method of claim 10, wherein said anti-IL-17 antibody or fragment thereof specifically binds to IL-17A or IL-17F or IL-17A and IL-17F.

14.-15. (canceled)

16. The method of claim 13, wherein said antibody or fragment thereof is a monoclonal antibody.

17.-18. (canceled)

19. The method of claim 16, wherein said antibody or fragment thereof is a human, a humanized or a chimeric antibody.

20.-21. (canceled)

22. The method of claim 1, wherein the IL-17 antagonist or IL-17 antibody or fragment thereof decreases mean vascular density in said tumor as compared to a tumor in a human subject that was not administered an effective amount of an IL-17 antagonist or IL-17 antibody or fragment thereof.

23.-24. (canceled)

25. The method of claim 22, wherein said inhibition of tumor angiogenesis decreases mean vascular density in said tumor in said human subject by at least 5%, or at least 10%, or at least 15%, or at least 20%, or at least 25%, or at least 30%, or at least 35%, or at least 40%, or at least 45%, or at least 50%, or at least 55%, or at least 60%, or at least 65% or at least 70%, or at least 75%, or at least 80%, or at least 85%, or at least 90%, or at least 95% or at least 99%, as compared to a tumor in a human subject that was not administered an effective amount of an IL-17 antagonist or IL-17 antibody or fragment thereof.

26.-27. (canceled)

28. The method of claim 25, wherein said mean vascular density is measured by taking an average area of CD31-positive cells in said tumor over a total area of cells in said tumor.

29.-34. (canceled)

35. The method of claim 1, further comprising administering to said human subject an anti-VEGF antibody or fragment thereof.

36.-37. (canceled)

38. The method of claim 35, wherein the anti-VEGF antibody is bevacizumab comprising a variable heavy chain sequence of SEQ ID NO: 1 and a variable light chain sequence of SEQ ID NO: 2, or fragment thereof.

39.-40. (canceled)

41. The method of claim 1, further comprising subjecting said human subject to chemotherapy or radiation therapy.

42.-43. (canceled)

44. The method of claim 1, wherein the tumor is in the colon, rectum, liver, lung, prostate, breast or ovary.

45.-46. (canceled)

47. The method of claim 1, further comprising monitoring the efficacy of said inhibition of tumor angiogenesis by determining the number or frequency of CD11b+Gr1+ cells in a tumor sample or in a peripheral blood sample obtained from said human subject, relative to the number or frequency of a tumor sample or peripheral blood sample obtained from said human subject prior to administration of said IL-17 antagonist.

48.-49. (canceled)

50. The method of claim 1, further comprising administering an effective amount of a G-CSF antagonist.

51.-52. (canceled)

53. The method of claim 50, wherein said G-CSF antagonist is an anti-G-CSF antibody or fragment thereof.

54.-61. (canceled)


SEQUENCE LISTING (8 sequences)

SEQ ID NO: 1 - protein, 123 aa, Artificial sequence [sequence omitted]

SEQ ID NO: 2 - protein, 108 aa, Artificial sequence
  humanized antibody variable light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 3 - protein, 10 aa, Mus musculus
GYTFTNYGMN

SEQ ID NO: 4 - protein, 17 aa, Mus musculus
WINTYTGEPTYAADFKR

SEQ ID NO: 5 - protein, 14 aa, Mus musculus
YPHYYGSSHWYFDV

SEQ ID NO: 6 - protein, 11 aa, Mus musculus
SASQDISNYLN

SEQ ID NO: 7 - protein, 7 aa, Mus musculus
FTSSLHS

SEQ ID NO: 8 - protein, 9 aa, Mus musculus
QQYSTVPWT